(19) United States
(12) Patent Application Publication
(10) Pub. No.: US 2025/0248799 A1
(43) Pub. Date: Aug. 7, 2025

(54) METHOD AND SYSTEM FOR FACIAL AUGMENTATION

(71) Applicant: GALDERMA HOLDING SA, Zug (CH)

(72) Inventor: Andreas NIKOLIS, Westmont (CA)

(73) Assignee: GALDERMA HOLDING SA, Zug (CH)

(21) Appl. No.: 19/187,558

(22) Filed: Apr. 23, 2025

Related U.S. Application Data
(63) Continuation of application No. PCT/IB2023/060735, filed on Oct. 24, 2023.
(60) Provisional application No. 63/419,216, filed on Oct. 25, 2022.

(51) Int. Cl. A61F 2/00; A61K 9/00; A61K 9/06; A61K 31/728; A61M 5/42

(52) U.S. Cl. CPC A61F 2/0059; A61K 9/0019; A61K 9/06; A61K 31/728; A61M 5/427

(57) ABSTRACT

A method for administering an injectable filler to compensate for an aesthetic deficit in the temporal region is provided. The method includes determining the volume deficit associated with loss of brow height requiring lifting or excess volume loss requiring volumization. The method includes selecting an injectable filler such as a hyaluronic acid gel filler selected from a group I filler or a group II filler. The hyaluronic acid gel is characterized by an X strain of 500% to 2500% and a G′ from 0 Pa to 300 Pa (group I) or an X strain of 0% to 500% and a G′ from 500 Pa to 800 Pa (group II). The method includes administering the group I filler as a deep injection to correct a deficit in lift or the group II filler as a deep injection or superficial injection to correct a deficit in volume.

### CROSS-REFERENCE TO RELATED APPLICATION

The present application is a continuation of International Application No. PCT/IB 2023/060735, filed Oct. 24, 2023, which claims priority to U.S. Provisional Application No. 63/419,216 filed on Oct. 25, 2022, the entire contents of each of which are incorporated herein by reference.

### FIELD

The present disclosure pertains to augmentative techniques using facial injections, and to reducing adverse effects associated with such injections.

### BACKGROUND

Changes of the skin, such as sagging, wrinkles, temporal volume loss, fine lines, and related issues, are often a result of facial aging. Various treatment options are available to mitigate such changes. These treatment options may include injections of various products, such as dermal fillers, into a portion of a subject's skin.

### SUMMARY

Temporal volumizing is an injection procedure that aims to restore lost volume in the temporal region to re-establish facial shape.

Studies have investigated the safety of injection procedures in which fillers or other products are injected into facial regions. Because many facial regions are highly vascularized, adverse events due to intravascular injections or vessel compression are of concern during these procedures.

The present disclosure addresses techniques and approaches for carrying out various facial injection procedures while mitigating adverse effects.

At least one embodiment of the present disclosure relates to a method for administering an injectable filler to correct an aesthetic deficit in the temporal region of a face. The method includes determining the aesthetic deficit in the temporal region of a face in which the aesthetic deficit is one of a deficit in lift or a deficit in volume. The method includes selecting an injectable filler in which the injectable filler is a hyaluronic acid gel filler selected from one of a group I filler or a group II filler. The group I filler includes hyaluronic acid gel characterized by strain along an x-axis direction (as may be referred to as xstrain, X-strain, or x-strain) of approximately 500% to approximately 2500% and an elastic modulus, G′, from approximately 0 Pa to approximately 300 Pa. The strain corresponds to the extent of deformation and the elastic modulus corresponds to elasticity/firmness of the material. The group II filler includes hyaluronic acid gel having an X strain of approximately 0% to approximately 500% and a G′ from approximately 500 Pa to approximately 800 Pa. See e.g., Åke Öhrlund, “Evaluation of Rheometry Amplitude Sweep Cross-over Point as an Index of Flexibility for HA Fillers,” 8 J. Cosmetics, Dermatological Sci. and Applns., 2, 2018, pp. 47-54, https://doi.org/10.4236/jcdsa.2018.82008, published Jun. 21, 2018. The method includes administering the determined group I filler to correct the determined aesthetic deficit, e.g., in the temporal region of a face, of a deficit in lift or administering the determined group II filler to correct the determined aesthetic deficit in volume.

In at least one embodiment, the group II filler comprises hyaluronic acid gel characterized by an X-strain of about 750% to about 1600% and G′ of about 10 Pa to about 200 Pa.

In at least one embodiment, the group II filler comprises hyaluronic acid gel characterized by an X strain of about 800% to about 1000% and G′ from about 100 Pa to about 200 Pa to correct the determined aesthetic deficit in volume and wherein the injection is a superficial injection.

In at least one embodiment, the group II filler comprises hyaluronic acid gel characterized by an X strain of about 1400% to about 1600% and G′ from about 10 Pa to about 100 Pa. The method includes injecting the group II filler to correct the determined aesthetic deficit in volume, wherein the injection is a deep injection.

In at least one embodiment, the group I fillers comprise hyaluronic acid gel characterized by an X strain of about 0% to about 100% and G′ from about 500 Pa to about 600 Pa.

In at least one embodiment, the deep injection is close to the periosteum and the superficial injection is between skin (layer 1) and subcutaneous fat (layer 2).

At least one embodiment of the present disclosure relates to a method allowing for a quantitative assessment to be carried out relating to an injection procedure. The method includes receiving, by a user input to a user interface, information of a subject. The method further includes identifying injection factors for injection, wherein the injection factors comprise: (i) a facial region of the subject to be modified; (ii) an aesthetic deficit in the facial region to be modified, wherein the aesthetic deficit is selected from one of lift and volume; and (iii) severity of the aesthetic deficit, wherein the severity is selected from one of mild, moderate, and severe. The method further includes identifying, based on the identified aesthetic deficit and the identified severity of the aesthetic deficit, a hyaluronic gel filler; determining, based on the at least one of the identified injection factors, a landmark of the facial region for injection, wherein the landmark of the facial region for injection comprises a reduced proximity to a blood vessel or a nerve; and providing an indication of the landmark for injection.

In at least one embodiment, the method includes providing an indication to inject the hyaluronic acid gel filler close to the periosteum or between skin (layer 1) and subcutaneous fat (layer 2).

In at least one embodiment, the method includes providing indication to inject the hyaluronic acid gel filler close to the periosteum is associated with a deficit in volume or a deficit in lift.

In at least one embodiment, providing the indication to inject the hyaluronic acid gel filler between skin and subcutaneous fat is associated with a deficit in volume.

In at least one embodiment, the method includes providing an indication of the identified hyaluronic acid gel filler.

In at least one embodiment, the facial region of the subject to be modified comprises at least one of temporal hollowing, skin lines, scarring of the skin, or skin folds.

At least one embodiment of the present disclosure relates to a non-transitory computer readable medium storing instructions that, when executed by one or more processors, cause the one or more processors to perform a method. The method includes receiving, by a user input to a user interface, information of a subject. The method includes identifying injection factors for injection, wherein the injection factors comprise: (i) a facial region of the subject to be modified, (ii) an aesthetic deficit in the facial region to be modified, wherein the aesthetic deficit is selected from one of lift and volume, and (iii) severity of the aesthetic deficit, wherein the severity is selected from one of mild, moderate, and severe. The method includes identifying, based on the identified aesthetic deficit and the identified severity of the aesthetic deficit, a hyaluronic gel filler. The method includes determining, based on the at least one of the identified injection factors, a landmark of the facial region for injection, wherein the landmark of the facial region for injection comprises a reduced proximity to a blood vessel or a nerve. The method includes displaying an indication of the landmark for injection.

In at least embodiment, the hyaluronic acid gel filler is one of a group I filler comprising hyaluronic acid gel characterized by an X strain of about 500% to about 2500% and a G′ from about 0 Pa to about 300 Pa; and a group II filler comprising hyaluronic acid gel having an X strain of about 0% to about 500% and a G′ from about 500 Pa to about 800 Pa.

In at least embodiment, the group II filler comprises hyaluronic acid gel characterized by an X strain of about 750% to about 1600% and G′ of about 10 Pa to about 200 Pa.

In at least embodiment, the group II filler comprises hyaluronic acid gel characterized by an X strain of about 800% to about 1000% and G′ from about 100 Pa to about 200 Pa to correct the determined aesthetic deficit in volume and wherein the injection is a superficial injection.

In at least embodiment, the group II filler comprises hyaluronic acid gel characterized by an X strain of about 1400% to about 1600% and G′ from about 10 Pa to about 100 Pa to correct the determined aesthetic deficit in volume and wherein the injection is a deep injection.

In at least embodiment, the group I fillers comprise hyaluronic acid gel characterized by an X strain of about 0% to about 100% and G′ from about 500 Pa to about 600 Pa.

In at least embodiment, the method further comprises displaying an indication to inject the hyaluronic acid gel filler on the periosteum.

In at least embodiment, the indication to inject the hyaluronic acid gel filler on the periosteum is associated with a deficit in volume or a deficit in lifting overlying tissues.

In at least embodiment, the indication to inject the hyaluronic acid gel filler between skin and subcutaneous tissues is associated with a deficit in volume.

In at least embodiment, the method further comprises displaying an indication of the identified hyaluronic acid gel filler.

In at least embodiment, the facial region of the subject to be modified comprises at least one of temporal hollowing, skin lines, scarring of the skin, or skin folds.

### BRIEF DESCRIPTION OF THE DRAWINGS

FIG. 1 shows a functional block diagram of a system for determining injection targets according to one embodiment.

FIG. 2 is a flow diagram showing a method of determining injection targets using the system of FIG. 1.

FIG. 3 is a graphical representation showing various outputs of the system of FIG. 1.

FIG. 4A is a graphical representation showing flexibility (xStrain) for a number of OBT™ and NASHA™ products.

FIG. 4B is a graphical representation showing firmness (G′) for a number of OBT™ and NASHA™ products.

FIG. 4C is a graphical representation showing flexibility (xStrain) for a number of products.

FIG. 4D is a graphical representation showing firmness (G′) for a number of products.

FIG. 5 is a graphical representation showing various injection targets.

FIGS. 6-8 are graphical representations showing various facial regions.

FIG. 9 is a tabular representation showing an experimental setup for investigating the safety and effectiveness of hyaluronic acid for the treatment of temporal hollows.

FIGS. 10 and 11 are sample images of patients throughout the experiment shown in FIG. 9.

FIG. 12 is a tabular representation of patient satisfaction for the experiment shown in FIG. 9.

FIG. 13A is a tabular representation of GA IS for the experiment shown in FIG. 9 and FIG. 13B is a graphical representation of the GAIS.

FIG. 14 is a tabular representation of volume deficit scale for the experiment shown in FIG. 9.

FIG. 15 is a tabular representation of improvement since baseline for the experiment shown in FIG. 9.

FIG. 16 is a tabular depiction of representative descriptors of the three-dimensional temporal volume increases observed at each visit, compared to Baseline for the experiment shown in FIG. 9.

FIG. 17 is a tabular representation of Temple Volume Deficit Scale (TVDS) at Baseline x hyaluronic acid volume used cross-tabulation for the experiment shown in FIG. 9.

FIG. 18 is a tabular representation showing an experimental setup for an example clinical trial for in vitro hyaluronic acid.

FIG. 19 shows the results of a second experiment for the clinical trial related to FIG. 18.

FIG. 20 shows the results of a second experiment for the clinical trial related to FIG. 18.

FIG. 21 shows the mean area for nine hyaluronic acid fillers for the clinical trial related to FIG. 18.

FIG. 22 shows two-dimensional renderings of the particles of a third experiment for the clinical trial related to FIG. 18.

FIG. 23 shows the particle size distributions for the clinical trial related to FIG. 18.

FIG. 24 is a tabular representation of swelling factor (ml/g) and capacity (%) of each filler for the clinical trial related to FIG. 18.

FIG. 25 is a tabular representation of a blinded review for sample related to the clinical trial related to FIG. 18.

FIG. 26 is a tabular representation of the results of the third experiment for the clinical trial related to FIG. 18.

FIG. 27 is a tabular representation of additional results of a third experiment for the clinical trial related to FIG. 18.

FIG. 28 is a tabular representation of a summary of findings of the clinical trial related to FIG. 18.

FIG. 29 is a tabular representation of alpha-numeric labels created for identifying each sample of the clinical trial related to FIG. 18.

FIG. 30A is an ultrasound image of patient's tissue before injection of OBT™.

FIG. 30B is an ultrasound image of patient's tissue after injection of OBT™.

FIG. 31A is an ultrasound image of patient's tissue before injection of NASHA™.

FIG. 31B is an ultrasound image of patient's tissue after injection of NASHA™.

FIG. 32 is a representation of layers of tissue of a patient.

### DETAILED DESCRIPTION

The details of one or more embodiments of the subject matter described in this specification are set forth in the accompanying drawings and the description below. Other features, aspects, and advantages of the subject matter will become apparent from the description, the drawings, and the claims.

Injections of fillers and other products into one or more regions of the face can reduce the appearance of changes caused by facial aging in certain regions. For example, injecting fillers into the face can reduce or even minimize the appearance of sagging or hollowing skin and/or other facial features. Because many facial regions, such as the temporal region, are highly vascularized, adverse effects of intravascular product administration is of concern during these procedures. To date, common approaches for avoiding highly vascularized regions involve subjective assessments by a practitioner or provider (healthcare professional, clinician, aesthetic technician, etc.) as to the location to perform one or more injections. In addition to adverse effects that may bear on safety, there are other undesirable effects, such as palpable product and/or poor efficacy, which may arise from treatment that does not properly reconcile the type of product to be injected with patient-specific (subject-specific) factors such as the nature of the deficit to be treated.

Generally speaking, the present disclosure relates to systems, methods, and algorithms for determining an injection process or technique to reduce, minimize, or virtually eliminate the risk of adverse effects during skin augmentation. While the present disclosure is generally described in relation to skin lifting and volumizing, it should be noted that the systems, methods, and algorithms described herein may be applicable to various skin or facial changes including, but not limited to, wrinkles, scarring, fine lines, skin folding, etc.

By automatically identifying injection depths and planes to avoid one or more blood vessels, one or more arteries, one or more veins, or one or more nerves of a subject (also referred to herein as a patient), the systems and methods herein can provide an efficient, effective, and safe manner of determining an injection process for various products. Further, such identifications aid in the objective determination of an injection location, effectively allowing for a built-in perimeter or safety zone between the injection site and an area known to generally be highly vascularized. Various other technical benefits and advantages are described in greater detail below.

### Exemplary Systems and Techniques

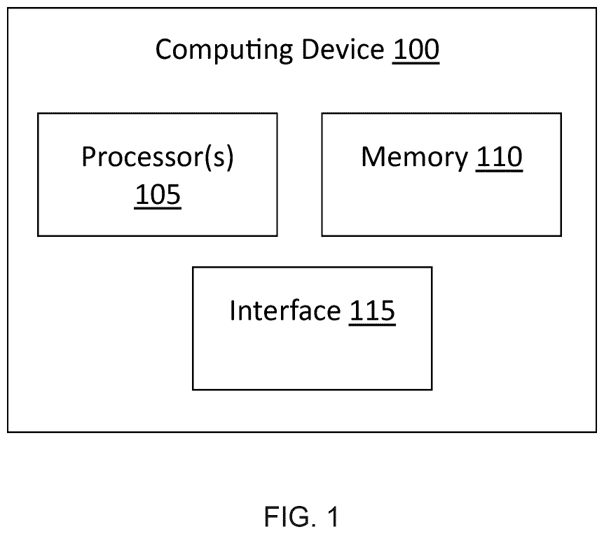

FIG. 1 shows an example computing device 100. The computing device 100 includes at least one processor 105, an interface 115, and memory 110. The memory 110 is configured to store machine instructions that, when executed by the processor 105, cause the processor 105 to perform operations to receive information regarding one or more facial regions of the patient and determine an injection target or process that is expected to have a reduced likelihood of adverse effects, as will be described in further detail below. The memory 110 may also store data to effect presentation of one or more resources, content items, measurements, etc., via the interface 115 (e.g., a display).

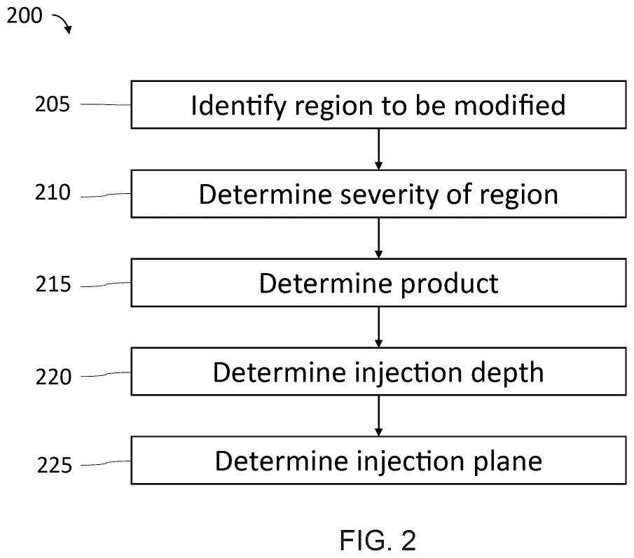

FIG. 2 shows a flow diagram of a method 200 used to determine one or more injection targets according to one embodiment. In at least one embodiment, one or more steps of the method 200 may be carried out by a non-transitory computer readable medium configured to store instructions which, when executed by a processor, cause particular operations to be carried out.

As shown in FIG. 2, in a step 205 of the method 200, the computing device 100 may be configured to receive a plurality of user inputs including information of a subject (e.g., a patient) to identify a region of the face, or other body part, to be modified. For example, the computing device 100 may be configured to receive the inputs via the interface 115 (e.g., one or more inputs to a display, touch screen, mouse click, keyboard input, or other inputs). The user inputs may indicate various information of a patient. For example, the computing device 100 may be configured to receive a plurality of measurements of a patient's face (e.g., during a clinical exam of the patient).

The measurements may include a distance between one or more facial features (e.g., a 3D topographic analysis of the temporal fossa and eyebrow position in relation to the orbital rim). For example, patient volumetric changes can be determined using normal photography where the changes in contour are quantified. Further, patient volumetric changes can be detected from an MRI, ultrasound, or another type of imaging system. For example, a high resolution 2D or 3D image capture system such as the Vectra® or Visia® systems (Canfield Scientific, Inc., Fairfield, NJ) may be used.

The measurements may include a distance between one or more facial features and one or more superficial landmarks on the skin. For example, the measurements may include a distance between a first portion of the face (e.g., a portion of an eye's bony orbit, a portion of a nose, a portion of an eyebrow, a portion of a mouth, etc.) and a superficial landmark of the face of the patient (e.g., the zygomaticofrontal suture line at the posterior orbital rim, the superior border of the zygomatic arch and lateral orbital rim junction, etc.). See, e.g., Nikolis, Andreas, et al., “Topography of the Deep Temporal Arteries and Implications for Performing Safe Aesthetic Injections.” Journal of Cosmetic Dermatology, vol. 21, no. 2, 2021, pp. 608-614., https://doi.org/10.1111/jocd.14672. The computing device 100 may be configured to flag (e.g., store in memory) the superficial landmarks of the patient to indicate the superficial landmarks are outside of a standard safety zone for injections.

The information of the patient may include various measurements of a portion of the face or other body parts determined by, for example, a facial ultrasound. The information of the patient may include additional and/or alternative information including, but not limited to, biometric data of the patient and medical history of the patient. The information of the patient may be stored in the memory 110. In some embodiments, the computing device 100 may be configured to automatically receive the plurality of inputs via one or more external devices (e.g., ultrasounds, scanners, cameras, sensors, or other devices) communicably coupled to the computing device 100 (e.g., via at least one network).

At step 210, the computing device 100 may be configured to identify injection factors for injection, wherein the injection factors comprise: (i) a facial region of the subject to be modified; (ii) an aesthetic deficit in the facial region to be modified, wherein the aesthetic deficit is selected from one of lift and volume; and (iii) severity of the aesthetic deficit, wherein the severity is selected from one of mild, moderate, and severe. These injection factors are discussed in more detail below.

At step 215, the computing device 100 may be configured to identify, based on the identified aesthetic deficit and the identified severity of the aesthetic deficit, a hyaluronic gel filler, and determine, at step 220, based on the at least one of the identified injection factors, a landmark of the facial region for injection. The landmark of the facial region for injection comprises a reduced proximity to a blood vessel or a nerve. For example, the reduced proximity may be a distance that is a threshold distance or less from an expected or actual location of a feature to be avoided, such as a blood vessel, artery, vein, or nerve. At step 225, the computing device 100 may be configured to provide (e.g., by displaying) an indication of the landmark for injection.

The computing device 100 may be configured to identify a region of the patient's face to be modified (e.g., augmented by treatment) based on the plurality of inputs. For example, the computing device 100 may be configured to identify the region based on the received measurements and/or one or more inputs to the interface 115 of the computing device 100. The region to be modified may include, but is not limited to, a temporal region, a forehead region, a chin region, an ear region, a cheek region, a nose region, or another portion of the face and/or body of the patient. For example, the identified region may include one or more changes in the skin and the deeper subcutaneous tissues such as fine lines, wrinkles, skin folds, volume loss, or another feature. In some embodiments, the computing device 100 may be configured to identify if the region is a target for lifting (e.g., tightening) of the skin or volumization (e.g., filling) of the skin. For example, lift is determined in mild to moderate volume loss of the temporal fossa, where the lateral brow has dropped from lack of overlying tissue support or structure. Volumizing is determined when the hollowing is significant in the region, yet the brow either does not extend laterally, or the deficit is so great that the primary outcome is volumization despite brow position. In some embodiments, a practitioner or provider (healthcare professional, clinician, aesthetic technician, etc.) may identify whether a particular region is a target for a particular treatment (e.g., lifting or tightening) and then provide input to a user interface (e.g., based on a scan of the patient's facial region or by manually indicating corresponding regions on a model).

Severity and improvement after treatment of a patient's skin can be detected and determined through one or more tools and/or scales. For example, severity can be based on the Galderma Temple Volume Deficit Scale @2019, Galderma Holding SA, Zug, Switzerland. Severity can be determined to be minimal (e.g., scale at 0) when no to minimal volume deficit is determined (e.g., no concavity, no bony demarcation visible). Severity can be mild (e.g., scale at 1) when mild volume deficit is determined (e.g., minimal to mild concavity, no to minimal bony demarcation). Severity can be moderate (e.g., scale at 2) when moderate volume deficit is determined (e.g., moderate concavity, visible bony demarcation). Severity can be high (severe) (e.g., scale at 3) when substantial volume deficit is determined (e.g., severe concavity, clearly apparent bony demarcation).

The computing device 100 may be configured to determine a severity of the region to be modified. For example, the computing device 100 may be configured to determine the severity based on the information of the patient and a threshold level. If the information (e.g., measurements) is below a first threshold (e.g., less than 10% concavity of temporal region, etc.) the computing device 100 may be configured to determine the severity of the region is mild. If the information (e.g., measurements) is above the first threshold, the computing device 100 may be configured to determine the severity of the region is moderate to severe, as another example.

The computing device 100 may be configured to determine a product to be used for the region to be modified based on the information of the patient and the determined severity of the region. For example, the computing device 100 may be configured to determine between various types of hyaluronic acid fillers including Non-Animal Stabilized Hyaluronic Acid (NASHA™, Galderma Holdings SA, Zug, Switzerland) and Optimal Balance Technology (OBT™ is also referred to as X presHA n Technology™, Galderma Holdings SA, Zug, Switzerland). For example, if it is determined that skin hollowing is the primary treatment concern for a patient, the computing device 100 may be configured to determine using OBT™ as the injection product. As another example, if it is determined that skin sagging is the primary concern to be treated for a patient, the computing device 100 may be configured to determine using NASHA™ as the injection product.

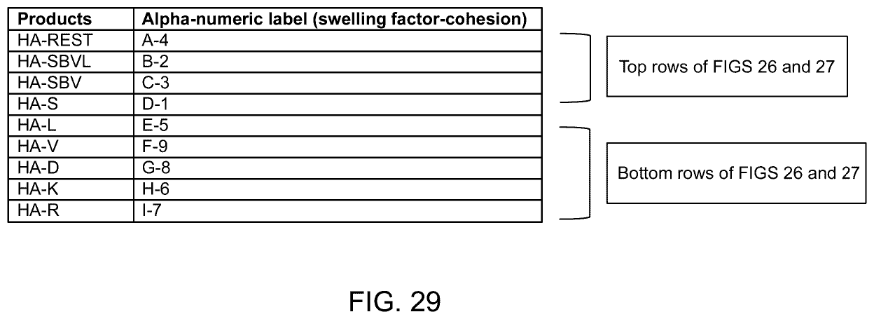

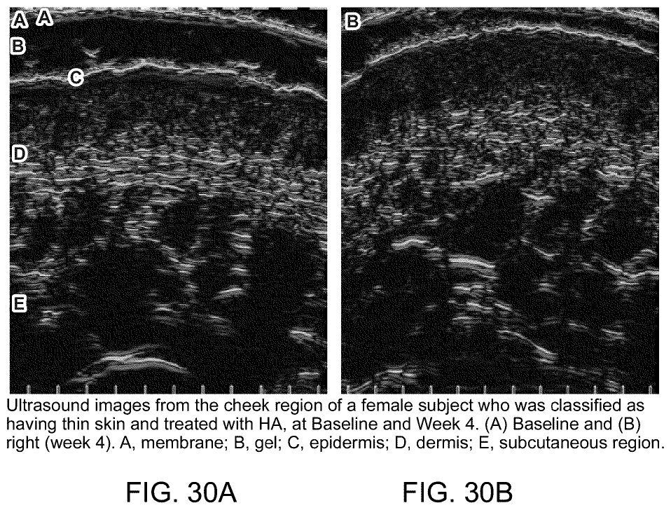

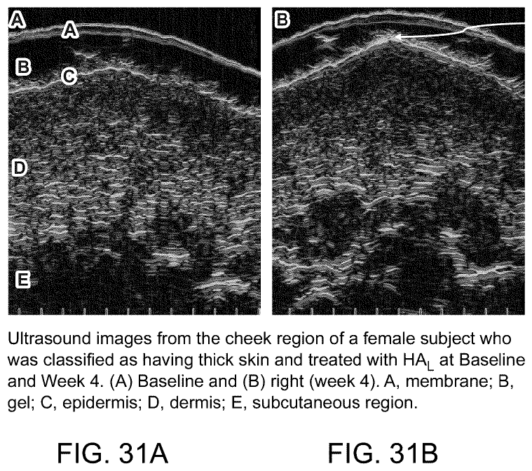

FIG. 30A shows an ultrasound image of a portion of tissue without products, and FIG. 30B shows an ultrasound image of a portion of tissue with OBT™. FIG. 31A shows an ultrasound image of a portion of tissue without products, and FIG. 31B shows an ultrasound image of a portion of tissue with NASHA™. NASHA™ integrates more slowly into surrounding tissues and as such allows the product to remain where it is placed (e.g., to form a peak 3100 shown in FIG. 31B) so as to realize the greatest lifting or projection potential. OBT™ generally integrates into the soft tissues more readily and as such corrects the volume deficit while maintaining a fuller yet natural appearance (e.g., integrates into the tissue without forming a peak as shown in FIG. 30B).

The various types of hyaluronic acid fillers including NASHA™ and OBT™ can include Restylane® FY NESSE (Galderma Holdings SA, Zug, Switzerland), Restylane® REFY NE (Galderma Holdings SA, Zug, Switzerland), Restylane® VOLY ME™ (Galderma Holdings SA, Zug, Switzerland), Restylane® KY SSE™ (Galderma Holdings SA, Zug, Switzerland), Restylane® DEFY NE (Galderma Holdings SA, Zug, Switzerland), Restylane® LY FT (Galderma Holdings SA, Zug, Switzerland), and Restylane® or Restylane-L® (Galderma Holdings SA, Zug, Switzerland).

Although particular suitability is subject to practitioner determination, Restylane® FY NESSE is generally utilizable for superficial wrinkles, Restylane® REFY NE is generally utilizable for mid-to-deep dermis injection for correction of moderate to severe facial wrinkles and folds, Restylane® VOLYME™ is generally utilizable for restoring facial volume along the cheeks, midface, chin and jawline, Restylane® KY SSE™ is generally utilizable for adding volume, smoothing upper lip lines, and enhancing lip color, Restylane® DEFY NE is generally utilizable for mid-to-deep dermis injection for correction of moderate to severe facial wrinkles and folds, Restylane® LY FT is generally utilizable for providing fullness to cheeks and adding volume to correct and smooth smile lines, and Restylane® is generally utilizable to add volume and fullness to the skin to correct moderate to severe facial wrinkles and folds. Aspects of these products are described in greater detail herein. It should be appreciated, however, that these products are provided by way of example and not of limitation, and that the techniques set forth herein are applicable to other products and classes of products (particularly for dermal fillers) not limited to any particular manufacturer or supplier. Poly-L-Lactic acid (PLLA), for example, SCULPTRA Aesthetic® (Galderma Holding SA, Zug, Switzerland), fat, micro fat, or nanofat injections may also be delivered using the techniques described herein.

The computing device 100 may be configured to determine one or more specific Restylane® products responsive to determining the region to be modified. For example, the computing device 100 may be configured to determine Restylane® LY FT should be used for mild to severe sagging. The computing device 100 may be configured to recommend (e.g., via a notification) not to inject Restylane® LY FT close to a temporal fusion line.

The computing device 100 may be configured to determine Restylane® REFY NE should be used for mild hollowing. The computing device 100 may be configured to determine Restylane® VOLY ME should be used for moderate to severe hollowing. The computing device 100 may be configured to recommend (e.g., via a notification) that Restylane® REFY NE and VOLY ME can be injected close to the temporal fusion line.

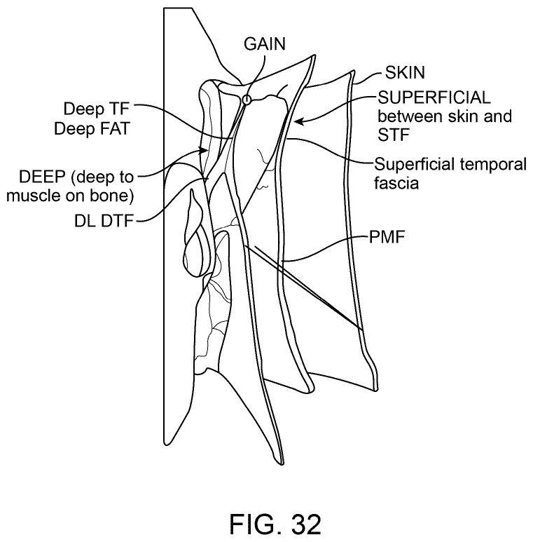

The computing device 100 may be configured to determine an injection depth or level of a needle to inject the product into the region to be modified based on the information, severity, and determined product. This determined depth may be communicated to the user as a target depth via the user interface. For example, the computing device 100 may be configured to determine between a superficial injection depth (e.g., above the superficial fascia as shown in FIG. 32) and a deep injection depth (e.g., below the superficial fascia as shown in FIG. 32). The computing device 100 may be configured to analyze the received information of the patient (e.g., the one or more measurements of the patient) and compare the information to the one or more identified superficial landmarks of the patient to determine the injection depth. The computing device 100 may be configured to determine the injection depth to avoid contact with deep temporal arteries blood vessels, nerves, and veins that support the eye and mastication muscles of the face to mitigate adverse effects during injection. For example, a relatively superficial injection depth (e.g., a first depth) may include one or more top layers of skin (e.g., a portion of the epidermis, a portion of the dermis, etc.). A relatively deep injection depth (e.g., a second depth) may include a deeper layer of skin or a layer beneath the skin (e.g., deep to the temporal region of the skin, such as subcutaneous fat, muscle or even the deepest at the periosteum, etc.). Injecting products at these depths can facilitate avoiding various adverse effects including, but not limited to, hematoma, vascular occlusion, necrosis, blindness, and nerve damage.

In some embodiments, the computing device 100 may be configured to receive one or more inputs indicating that brow ptosis is present. Responsive to receiving the input, the computing device 100 may be configured to determine that Restylane® LY FT should be injected at a relatively deep injection depth.

The computing device 100 may be configured to determine an injection plane of a needle to inject the product into the region to be modified to mitigate adverse effects during injection. For example, the computing device 100 may be configured to determine a first injection which is a deep injection, e.g., one that is performed at a relatively deeper depth (such as a depth beyond or exceeding a predetermined depth) that can be deeper than a second injection performed at a relatively shallower depth than the first injection. The deep injection may be referred to as an injection at depth.

In at least one embodiment, the injection at depth is performed deeper than a predetermined depth that may be a superficial depth corresponding to a superficial layer between skin and subcutaneous fat of the patient. In at least one embodiment, the computing device 100 may be configured to determine a first injection plane defined at the periosteum (e.g., at a first layer) of the skin responsive to determining a deep injection depth is adequate. In at least one embodiment, the first and second injections may be performed at a substantially equivalent depth. In at least one embodiment, the computing device 100 may be configured to determine that aspiration is recommended to avoid intravascular injection of deep tissue arteries, blood vessels or nerves.

In at least one embodiment, the computing device 100 may be configured to determine a second injection plane defined between a layer of the skin and a layer of subcutaneous fat (e.g., layer 2) responsive to determining that a superficial injection depth is adequate. Thus, in at least one embodiment, the deep injection is close to (that is, proximate to) the periosteum and the superficial injection is between skin (layer 1) and subcutaneous fat (layer 2). In these embodiments, for the superficial injection, the computing device 100 may be configured to determine the plane is defined at a junction of ligaments into a superficial fatty layer of skin.

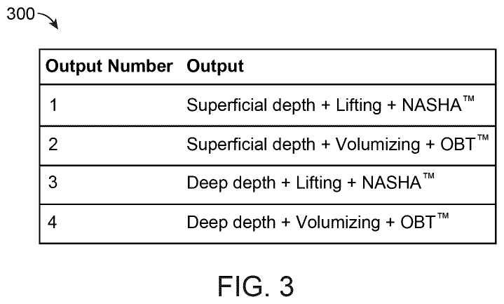

The computing device 100 may be configured to output guidance for performing an injection process tailored to a patient via the interface 115 of the computing device 100 (e.g., via a display). For example, the injection process may include one or more of at least four outcomes based on the determined region and aesthetic treatment concern and the severity. Representative outcomes are shown in the table 300 in FIG. 3.

When the computing device 100 determines that lifting of the skin is a potential modification of the identified region, the computing device 100 may be configured to output the first output 1 to indicate that the target injection depth is superficial and the product includes NASHA™. When the computing device 100 determines that lifting of the skin is a potential modification of the identified region, the computing device 100 may be configured to output the third output 3 to indicate that the target injection depth is deep and the product includes NASHA™.

When the computing device 100 determines that volumizing of the skin is a potential modification of the identified region, the computing device 100 may be configured to output the second output 2 to indicate that the target injection depth is superficial and the product includes OBT. When the computing device 100 determines that volumizing of the skin is a potential modification of the identified region, the computing device 100 may be configured to output the fourth output 4 to indicate that the target injection depth is deep and the product includes OBT.

While FIG. 3 displays various possible outputs, the computing device 100 may be configured to output various information. For example, the computing device 100 may be configured to output various guidance, notifications, recommendations, suggestions, or other components based on the determined injection target. The computing device 100 may be configured to display the injection target through one or more images, videos, sounds, or texts. For example, the computing device may be configured to output “inject product A at B mm from landmark C,” where “A” is the determined product, “B” is a determined distance between the identified superficial landmark and a measured feature of the patient's face, and “C” is the identified landmark. It should be understood that this example is for illustrative purposes. The output can include various audio or visual information relating to the determined injection target. Such output may take the form of guidance, such as guidance for an injection depth and/or location, so as to inform the practitioner of a given injection site and/or region.

In particular, in at least one embodiment, the noted output may take the form of audiovisual indications to a practitioner. For example, a method according to at least one embodiment may include providing an indication to inject the hyaluronic acid gel filler proximate to the periosteum or between skin (layer 1) and subcutaneous fat (layer 2). In at least one embodiment, the indication to inject the hyaluronic acid gel filler close to the periosteum is associated with a deficit in volume or a deficit in lift. In at least one embodiment, the indication to inject the hyaluronic acid gel filler between skin and subcutaneous fat is associated with a deficit in volume. In at least one embodiment, an indication of the identified hyaluronic acid gel filler is provided. In at least one embodiment, an option for selection of the identified hyaluronic acid gel filler or another available hyaluronic acid gel filler is provided, e.g., through a display interface.

A practitioner may inject the determined product at the determined injection plane and depth based on the determined region to be modified and the modification. For example, the practitioner may inject the product into the patient's face using a standard injection needle (e.g., a 27G×1/2 needle). In at least some embodiments, the region to be modified includes one or more of temporal hollowing, skin lines, scarring of the skin, or skin folds.

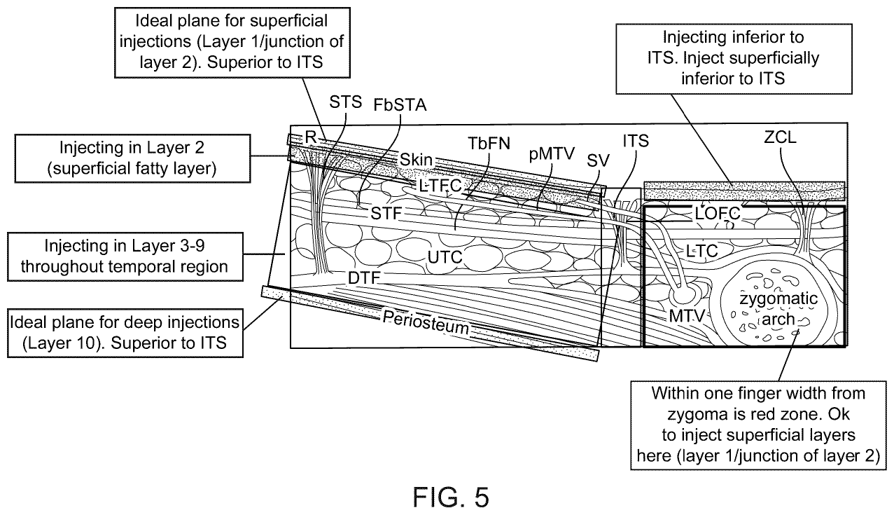

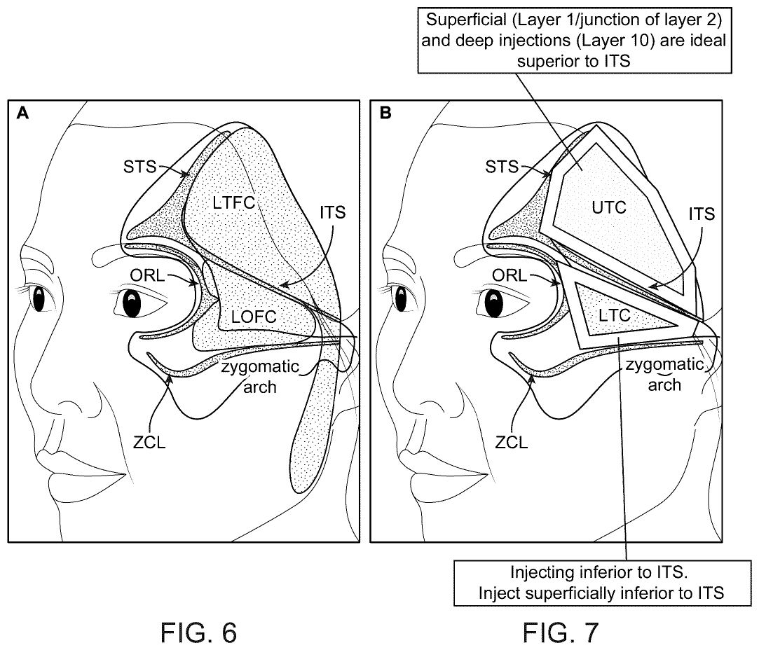

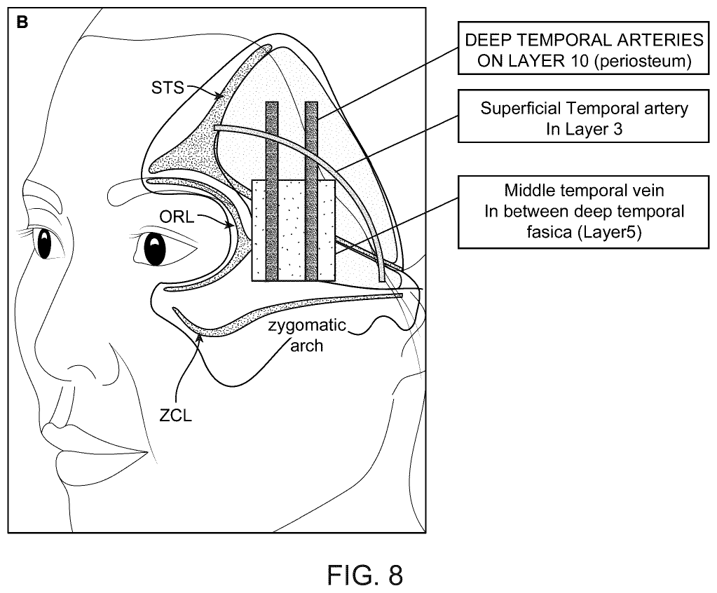

FIG. 5 shows various planes and regions of a subject's temporal region and FIGS. 6-7 show various graphical representations of portions of a subject's temporal region including potential injection areas and superficial landmarks. For example, as shown in FIG. 7, if the region to be modified is determined superior to the inferior temporal septum (“ITS”), the computing device 100 may be configured to determine superficial and deep injections are to be targeted to mitigate adverse effects. The computing device 100 can depict one or more regions and one or more of a plurality of layers making up the region. The computing device 100 can provide an instruction, for example, to take caution in injecting a region inferior to the IT C, and to only inject a region superficially inferior to the ITS. Further, within a finger's width distance from the zygomatic arch, a zone can be graphically highlighted by the computing device 100, which can output an instruction to only inject superficial layers in that area. In particular, various guidance can be outputted, e.g., audiovisually, by the computing device 100, so as to instruct a user to take caution in injecting Layer 2 (a superficial fatty layer) and to avoid injecting Layers 3-9 entirely throughout the temporal region. As another example, if the region to be modified is determined inferior to the ITS, the computing device 100 may be configured to determine that superficial injections should be performed to mitigate adverse effects. The computing device 100 can further provide a cautionary notification regarding injecting the region inferior to the ITS shown in FIG. 7 and can provide a notification indicating that injection should only be performed in an area superficially inferior to the ITS. FIG. 8 shows a graphical example of various temporal arteries to be avoided during an injection process.

### Product Characterization

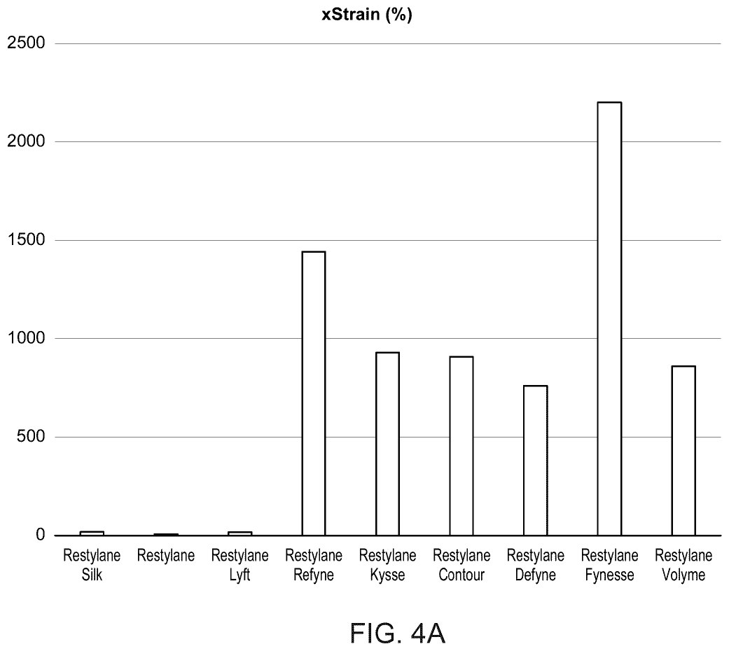

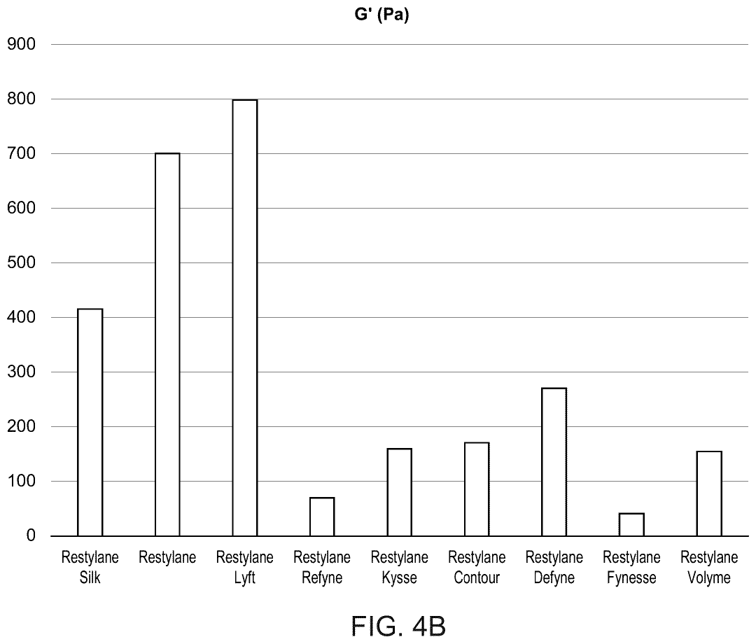

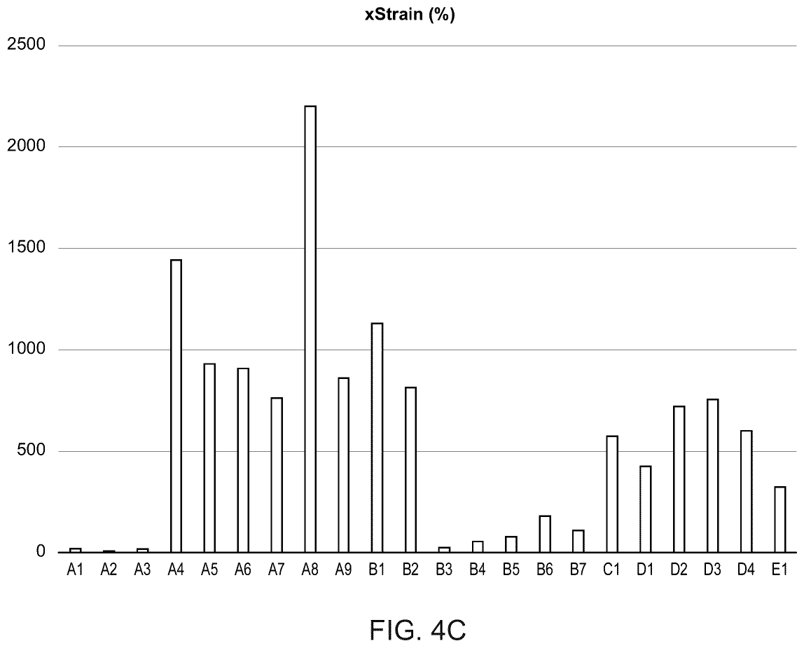

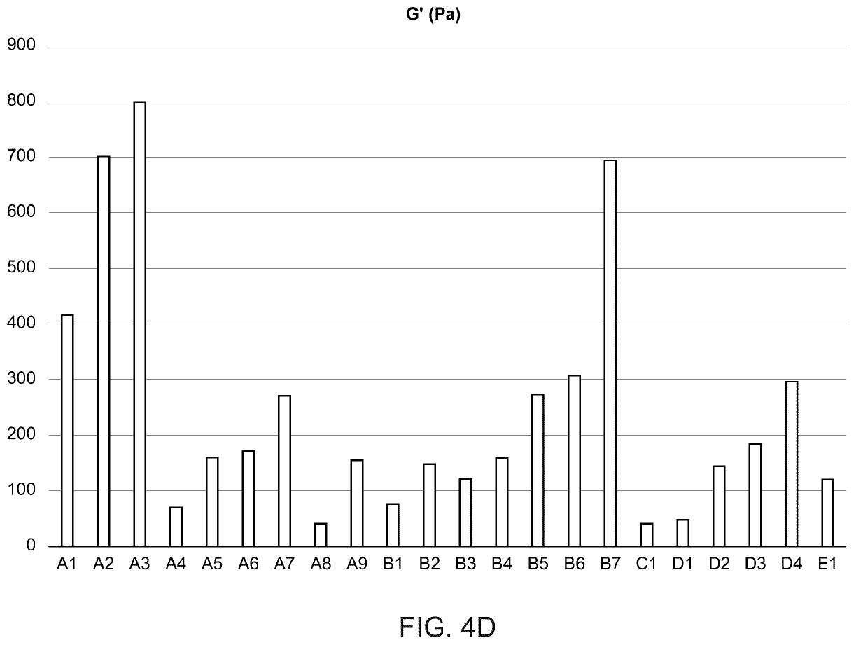

FIGS. 4A and 4B show graphical representations of various NASHA™ and OBT™ products in terms of flexibility (X strain, %) and firmness (G′, Pa). See, e.g., Öhrlund, Å ke, “Evaluation of Rheometry Amplitude Sweep Cross-over Point as an Index of Flexibility for HA Fillers,” 8 J. Cosmetics, Dermatological Sci. and Applns., 2, 2018, pp. 47-54, https://doi.org/10.4236/jcdsa.2018.82008, published Jun. 21, 2018.)

The firmness of a product, measured as G′ using rheometry, is performed under nearly static conditions. The deformations used in these measurements are small, in order to keep within the linear viscoelastic region (LVR), the region where the stress changes linearly with deformation. These measurements can be performed as a frequency sweep. In order to determine a suitable level of deformation to use in the frequency sweep, an amplitude sweep is performed, where the amount of deformation is increased until a change in the results is observed, indicating the end of the LVR. A level of deformation where the measured firmness is stable is chosen for the frequency sweep.

A point that can be identified precisely is the cross-over point, where G′ and G″ intersect.

At this cross-over point, the strain can be evaluated as a measure of flexibility. A material with a large xStrain can stand a large deformation before yielding, and can therefore be considered to be more stretchable, or flexible. The cross-over strain value may be considered a flexibility index for the material. The OBT™ family of products has previously been found to cover a large span of G′ values as measured from a frequency sweep at small deformations.

The rheology measurement was performed in a sequence including a relaxation time of 30 min, a frequency sweep from 10 to 0.1 Hz at 0.1% strain, followed by an amplitude sweep from 0.1 to 10000% (0.001 to 100) strain at 1 Hz. The gap was 1 mm using a PP25 measuring system at 25° C. (e.g., a 25 mm measuring plate of a rheometer such as the MCR 302 rheometer from Anton Paar of Graz, Austria). The frequency sweep was evaluated for G′, G″, G* and tan delta at 0.1 Hz. The amplitude sweep was first evaluated at 0.1% strain in order to verify that the applied frequency sweep strain was within the linear viscoelastic range. Secondly, the strain was evaluated at the cross-over point of the amplitude sweep, i.e., the point where G′ and G′ have the same value.

Further techniques for evaluating flexibility of fillers are set forth in U.S. Patent Application Publication No. 2020/0072721 entitled “Flexibility Measurements of Injectable Gels,” published Mar. 5, 2020, which is incorporated by reference herein in its entirety for the methodologies and process disclosed therein.

NASHA™ and OBT™ products can be grouped into at least two groups of fillers. Group I (e.g., OBT™ products) can include products characterized by a flexibility (X strain) of approximately 500% to approximately 2500% and a firmness (G′) of approximately 0 to approximately 300 Pa.

For example, as shown in FIGS. 4A and 4B, Group I products can include Restylane® FY NESSE (approximately 2000%-approximately 2500% X strain, approximately 0—approximately 100 Pa G′), Restylane® REFY NE (approximately 1200%-approximately 1600% X strain, approximately 0-approximately 100 Pa G′), Restylane® VOLY ME (approximately 600%-approximately 1200% X strain, approximately 100-approximately 200 Pa G′), Restylane® KY SSE (approximately 600%-approximately 1200% X strain, approximately 100—approximately 200 Pa G′), and Restylane® DEFY NE (approximately 500%-approximately 1100% X strain, approximately 200-approximately 300 Pa G′). In some embodiments, the Group I products (dermal fillers) include hyaluronic acid gel characterized by an X strain of approximately 0% to approximately 100% and G′ from approximately 500 Pa to approximately 600 Pa.

Group II (e.g., the NASHA™ products) can include products characterized by an X strain (indicative of flexibility) of approximately 0% to approximately 500% and an elastic modulus G′ (indicative of firmness) of approximately 500 to approximately 800 Pa. For example, Group II products can include Restylane® LY FT (approximately 0%-approximately 500% X strain, approximately 700-approximately 800 Pa G′ or approximately 500-approximately 600 Pa G′) and Restylane® (approximately 0%-approximately 500% X strain, approximately 500—approximately 600 Pa G′ or approximately 700-approximately 800 Pa G′).

In some embodiments, the Group II products can include hyaluronic acid gel characterized by an X strain of approximately 750% to approximately 1600% and G′ of approximately 10 Pa to approximately 200 Pa. In some embodiments, the Group II products include hyaluronic acid gel characterized by an X strain of approximately 800% to approximately 1000% and G′ from approximately 100 Pa to approximately 200 Pa. In some embodiments, Group II products including hyaluronic acid gel characterized by an X strain of approximately 800% to approximately 1000% and G′ from approximately 100 Pa to approximately 200 Pa are used to correct the determined aesthetic deficit in volume, in which the injection of the Group II products is a superficial injection.

In some embodiments, the Group II products include hyaluronic acid gel characterized by an X strain of approximately 1400% to approximately 1600% and G′ from approximately 10 Pa to approximately 100 Pa. In some embodiments, such products are provided to correct the determined aesthetic deficit in volume, in which the injection is a deep injection.

As described herein, the determined product can be administered by injection into the skin. Generally, the Group I fillers can correct the determined aesthetic deficit of a deficit in lift and the Group II fillers can correct the determined aesthetic deficit in volume.

FIGS. 4C-4D show graphical representations of various products in terms of flexibility (X strain, %) and firmness (G′, Pa). Certain products shown in FIGS. 4C and 4D (e.g., products A1-E1) can be Group I or Group II products, or alternative products. See, e.g., Å ke Öhrlund, “Evaluation of Rheometry Amplitude Sweep Cross-over Point as an Index of Flexibility for HA Fillers,” 8 J. Cosmetics, Dermatological Sci. and Applns., 2, 2018, pp. 47-54, https://doi.org/10.4236/jcdsa.2018.82008, published Jun. 21, 2018.

Product A1 can include hyaluronic acid gel characterized by an X strain of approximately 0% to approximately 100% and G′ of approximately 400 Pa to approximately 500 Pa. Product A 2 can include hyaluronic acid gel characterized by an X strain of approximately 700% to approximately 800% and G′ of approximately 0 Pa to approximately 100 Pa. Product A3 can include hyaluronic acid gel characterized by an X strain of approximately 750% to approximately 850% and G′ of approximately 10 Pa to approximately 100 Pa. Product A4 can include hyaluronic acid gel characterized by an X strain of approximately 0% to approximately 100% and G′ of approximately 1000 Pa to approximately 2000 Pa. Product A5 can include hyaluronic acid gel characterized by an X strain of approximately 100% to approximately 200% and G′ of approximately 900 Pa to approximately 1000 Pa. Product A6 can include hyaluronic acid gel characterized by an X strain of approximately 100% to approximately 200% and G′ of approximately 900 Pa to approximately 1000 Pa. Product A7 can include hyaluronic acid gel characterized by an X strain of approximately 200% to approximately 300% and G′ of approximately 700 Pa to approximately 800 Pa. Product A8 can include hyaluronic acid gel characterized by an X strain of approximately 0% to approximately 100% and G′ of approximately 2000 Pa to approximately 2500 Pa. Product A9 can include hyaluronic acid gel characterized by an X strain of approximately 100% to approximately 200% and G′ of approximately 800 Pa to approximately 900 Pa.

Product B1 can include hyaluronic acid gel characterized by an X strain of approximately 0% to approximately 100% and G′ of approximately 1000 Pa to approximately 1200 Pa. Product B2 can include hyaluronic acid gel characterized by an X strain of approximately 100% to approximately 200% and G′ of approximately 800 Pa to approximately 900 Pa. Product B3 can include hyaluronic acid gel characterized by an X strain of approximately 0% to approximately 100% and G′ of approximately 0 Pa to approximately 100 Pa. Product B4 can include hyaluronic acid gel characterized by an X strain of approximately 100% to approximately 200% and G′ of approximately 0 Pa to approximately 100 Pa. Product B5 can include hyaluronic acid gel characterized by an X strain of approximately 200% to approximately 300% and G′ of approximately 0 Pa to approximately 100 Pa. Product B6 can include hyaluronic acid gel characterized by an X strain of approximately 250% to approximately 350% and G′ of approximately 150 Pa to approximately 250 Pa. Product B7 can include hyaluronic acid gel characterized by an X strain of approximately 650% to approximately 750% and G′ of approximately 100 Pa to approximately 200 Pa.

Product C1 can include hyaluronic acid gel characterized by an X strain of approximately 0% to approximately 100% and G′ of approximately 500 Pa to approximately 600 Pa.

Product D1 can include hyaluronic acid gel characterized by an X strain of approximately 0% to approximately 100% and G′ of approximately 400 Pa to approximately 500 Pa. Product D2 can include hyaluronic acid gel characterized by an X strain of approximately 100% to approximately 200% and G′ of approximately 700 Pa to approximately 800 Pa. Product D3 can include hyaluronic acid gel characterized by an X strain of approximately 100% to approximately 200% and G′ of approximately 700 Pa to approximately 800 Pa. Product D4 can include hyaluronic acid gel characterized by an X strain of approximately 250% to approximately 350% and G′ of approximately 550 Pa to approximately 650 Pa.

Product E1 can include hyaluronic acid gel characterized by an X strain of approximately 100% to approximately 200% and G′ of approximately 300 Pa to approximately 400 Pa.

### Examples

The process as described herein for a method for administering an injectable filler to correct an aesthetic deficit in the temporal region of a face was applied in the following examples.

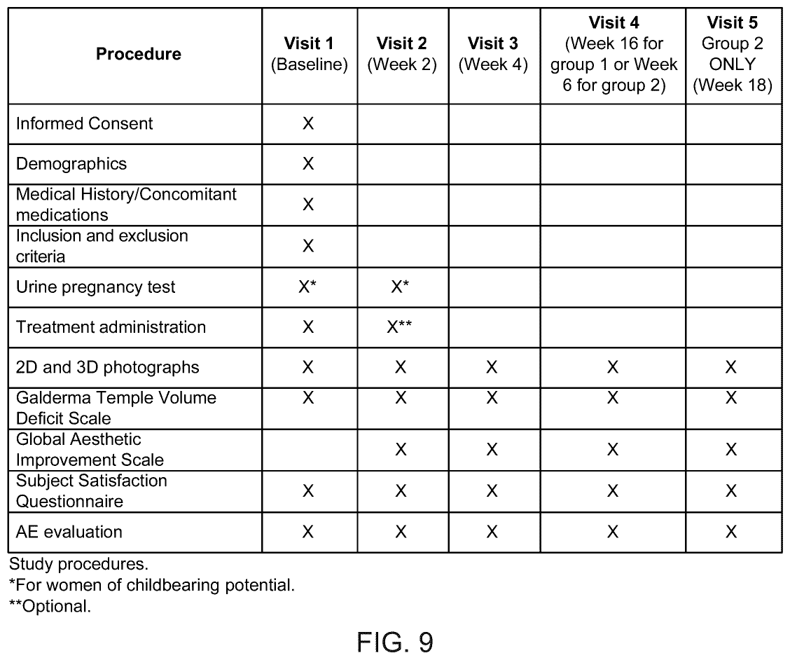

FIG. 9 is a tabular representation showing an example experimental setup for investigating the safety and effectiveness of HA-V for the treatment of temporal hollows. For example, hyaluronic acid (HA) fillers manufactured using OBT™ have distinctive physical properties that are advantageous in their use for specific indications. Product A9 (HA-V) has a large gel calibration (particle size), which may make it particularly suitable for volumizing large surface areas such as the temporal hollows.

In an example experiment, a prospective, open label, single cohort, clinical trial was conducted. Twenty-six females who presented with bilateral temporal hollows at baseline were recruited. All subjects received treatment with HA-V and were observed at 4 to 5 in-person visits over 16 weeks. Subjective and objective measures of safety and efficacy parameters were collected via 2- and 3-dimensional imagery, questionnaires/scales (i.e., subject satisfaction, global aesthetic improvement, temporal hollowing severity), and adverse event diaries.

To achieve targeted correction, the experimental example used an average of 3.40 syringes per subject. A II treatments were performed using a bolus injection technique to place the product on the periosteum (bone) of the temporal region. Following correction, all subjects (100%) displayed improvement in their global aesthetic appearance, and 25/26 (96.15%) displayed ≥1 grade improvement on the temporal volume scale. Subject satisfaction was high, with 91.3% of subjects being satisfied with the appearance of their temporal regions following correction. In this experiment, HA-V was evidenced to have an excellent safety profile and proven efficacy up to 16 weeks, making it a suitable HA filler for volumization of the temporal region.

In particular, during this example experiment, subjects having achieved desired correction following the first treatment: Visit 1: Screening/Baseline+Treatment 1 (Week 0), Visit 2a: Safety visit (Week 2), Visit 3: Follow-up 1 (Week 4 since correction), Visit 4: Follow-up 2 (Week 16 since correction). Subjects received two treatments: Visit 1: Screening/Baseline+/Treatment 1 (Week 0), Visit 2a: Safety visit+Treatment 2 (Week 2), Visit 2b: Safety visit (Week 4 since first treatment), Visit 3: Follow-up 1 (Week 4 since second treatment; Week 6 since first treatment), Visit 4: Follow up 3 (Week 16 since second treatment; Week 18 since first treatment).

Twenty-six (26) subjects were recruited, for a total of 52 bilateral observations. Inclusion criteria included: Female gender (if of childbearing potential, a negative urine pregnancy test before all treatments); being aged 21 to 70 years at baseline; having bilateral scores >1 on the Galderma Temple Volume Deficit Scale (GTVDS, Galderma Holding SA, Zug, Switzerland), as assessed by the treating investigator at baseline (note: subjects were not required to have symmetrical temporal hollowing at baseline); and provided a signed and dated informed consent form to participate in the study. Exclusion criteria included: Subjects presenting with a known allergy to HA fillers or amide local anesthetics; subjects presenting with porphyria or other liver diseases; subjects with active skin disease, such as inflammation, infection or tumors, in or near the intended treatment sites; subjects with bleeding disorders or subjects who are taking thrombolytics or anticoagulants; subjects using immunosuppressants; history of other treatment/procedure in the previous six months that, in the treating investigator's opinion, would interfere with the study injections and/or study assessments or expose the subject to undue risk by study participation; visible markings (e.g., tattoos, scars, piercings) that in the treating investigator's opinion, may interfere with results or assessments.

HA-V is a sterile, biodegradable, transparent gel of non-animal cross-linked HA with the addition of lidocaine hydrochloride 3 mg/mL to diminish the pain resulting from the injection during the treatment. The gel was supplied in a prefilled plastic syringe, packaged individually in a blister, with two 27G×1/2″ ultra-thin wall needles. Products were used prior to the expiration date printed on the package. They were stored at a temperature of up to 25° C. (77° F.), were not frozen or refrigerated, and were protected from sunlight.

HA-V is indicated for injection into the supraperiostic zone or subcutis to augment the volume of the cheeks. However, in the example experiment, HA-V was used in injections performed relatively deep, on the periosteum overlapping the temporal bone, for volumizing temporal hollows. Each subject was eligible to receive injection volumes of up to 2 ml per temporal hollow, per session (i.e., the maximum quantity listed in the product insert). Volumes used varied to some extent between subjects and visits and were limited to achieving anatomic and aesthetic correction. The two treatment sessions were separated by two weeks, with the touch-up treatment at Visit 2/Week 2 being optional if desired correction was not achieved following the first treatment. Treatments were performed using the needle provided in the manufacturer's packaging.

Study procedures performed at each visit are depicted in FIG. 9. Procedures performed at the baseline visit included: Informed consent, demographics, medical history, concomitant medications, urine pregnancy test (for women of childbearing potential), confirmation of inclusion and exclusion criteria, 3D and 2D pictures before treatment, subject satisfaction with the appearance of the temporal region before treatment, treatment with HA-V, live adverse event (AE) evaluation (investigator), and disbursement of the subject A E diary. Subjects completed their A E diary for 14 days following each treatment (as applicable) and returned them to site personnel at the following visits. If any signs and/or symptoms were reported by subjects as being ongoing at follow up visits (Day 14+), the treating investigator assessed the subject to determine if the event was to be considered an AE. At Visits 2 to 4/5, subjects completed a 5-point satisfaction questionnaire (i.e., the Subject Satisfaction Questionnaire (SSQ)), which rated overall level of satisfaction with treatment results from “extremely satisfied” to “extremely dissatisfied”. Following study completion, a blinded reviewer assessed all subject imagery using the four-point GTVDS, and five-point Global A esthetic Improvement Scale (GAIS). The GTVDS was used to rate the severity of temporal hollowing from none (0) to severe (3), and the GAIS was used to rate the degree of aesthetic improvement from “worse” to “very much improved”.

The primary endpoints were the establishment of safety and efficacy up to Week 16 following the last injection, via (i) assessment of adverse events, at all visits, and (ii) proportion of subjects achieving at least a one-point improvement on the GTVS, at the end of study visit (Visit 4 or 5, depending on number of treatments administered].

The secondary endpoints were the establishment of efficacy up to Week 16 following the last injection, via (iii) proportion of subjects achieving at least a one-point improvement on the GTVS, at all follow up visits following last treatment, (iv) percentage of subjects having improved (e.g., “improved”, “much improved” or “very much improved”) since baseline, as assessed by the GAIS, at all visits, and (v) percentage of subjects self-reporting being at least somewhat satisfied (e.g., “slightly satisfied”, “satisfied”, “extremely satisfied”) with results after treatment(s), at all visits, as per the SSQ.

A II (100%) subjects were female. The average subject age was 51.92 years (SD: 8.58; Range: 25 to 69). Ethnic subgroups included 23/26 (88.5%) Caucasian, 2/26 (7.7%) Latin American, and 1/26 (3.8%) A rab subjects. The average height and weight of subjects was 165.96 cm (SD: 7.52) and 134.69 lbs. (SD: 18.63), respectively. Over half of the subjects (15/26, 57.7%) were post-menopausal, 2/26 (7.7%) were peri-menopausal, and 9/26 (34.6%) were pre-menopausal.

A factorial ANOVA investigated the effect of visit (V1 or V2a) and side (right versus left) on the volumes of HA injected. It was found that there was a significant effect of visit (F (1)=140.878, p=<0.001), but not of side (F(1)=0.009, p=<0.924), on the volume of HA used. The average volume used for the first bilateral treatment (V1) was 2.80 mL (Std. Error: 0.085), and for the second bilateral treatment (V2a, as applicable) was 0.60 mL (Std. Error: 0.100). Eleven (11/26, 42.31%) subjects received a second treatment at V2a. Only these subjects were seen for a safety follow up two weeks later (V2b). To achieve desired correction, the investigator used an average of 3.40 syringes per subject.

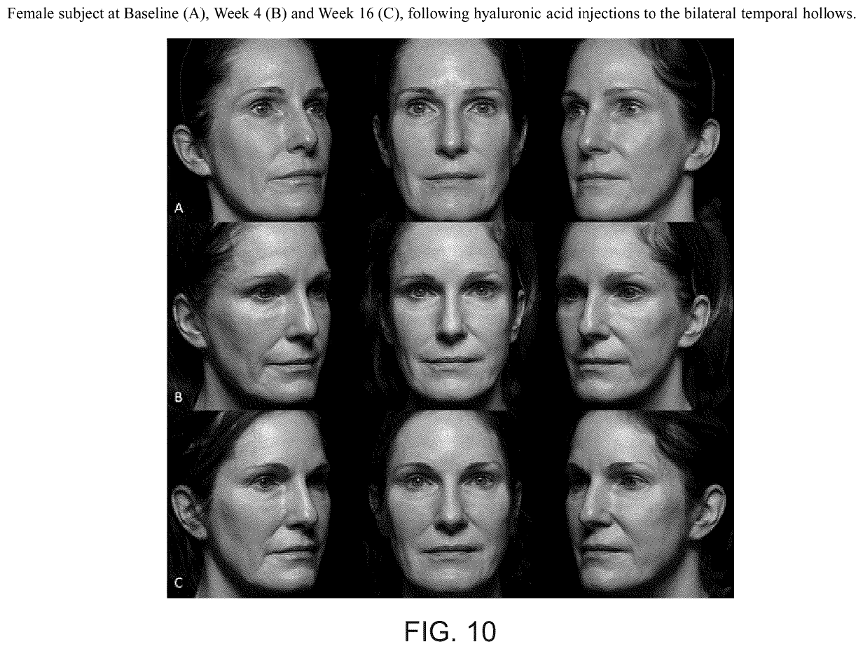

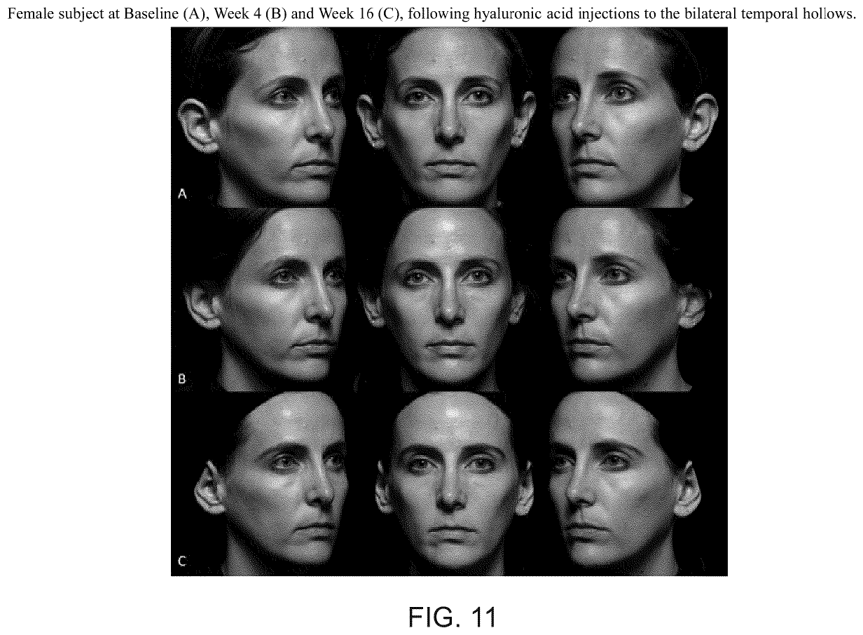

FIGS. 10 and 11 are sample images of patients throughout the experiment shown in FIG. 9. Aspiration was performed prior to all injections. There were no cases of a positive aspiration observed. A II treatments were performed using a bolus injection technique. Product was placed on the periosteum (bone) of the temporal region. Following treatment, all subjects remained onsite for a minimum of thirty minutes to observe for AEs. During the first ten minutes, they were instructed to apply light pressure to the injection sites, to diminish the likelihood of swelling or hematoma. Aside from injection-related AEs (e.g., entry point bleeding, redness), there were no immediate product-related AEs observed following treatment. Two subjects received a COVID-19 vaccine during the study follow up period. These subjects did not report swelling at or near the HA injection sites.

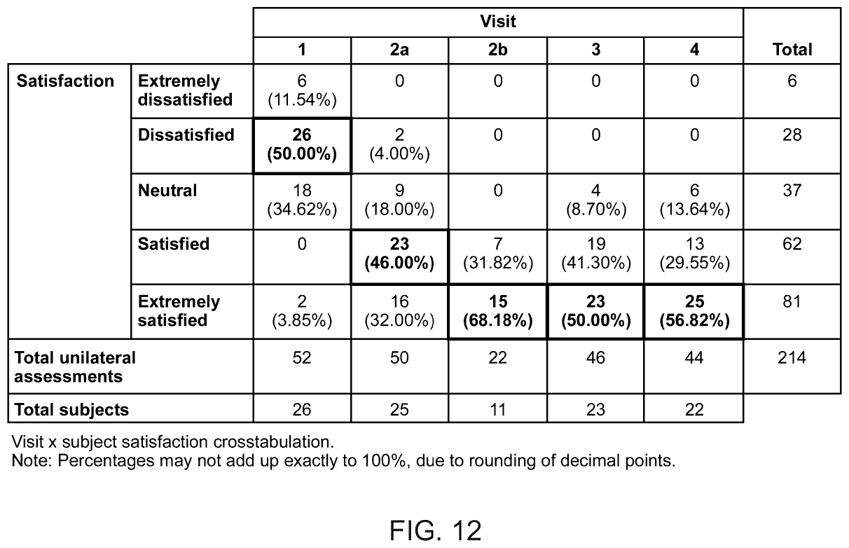

FIG. 12 is a tabular representation of patient satisfaction for the experiment shown in FIG. 9. Chi square tests revealed no significant relationship between side (right versus left) and satisfaction scores (p=0.969), therefore the data from lateral sides were averaged. Subsequent chi square tests revealed a significant effect of visit on subject satisfaction scores (p=<0.001, X² (16)=156.024), with frequency distributions having improved since Baseline. For example, most of subjects were dissatisfied or extremely dissatisfied with the appearance of their temporal region at Baseline (61.54%). After desired correction, 91.3% and 86.37% of subjects were satisfied or extremely satisfied with treatment results (V3 and V4, respectively; FIG. 12). There were no reports of subjects being dissatisfied following correction.

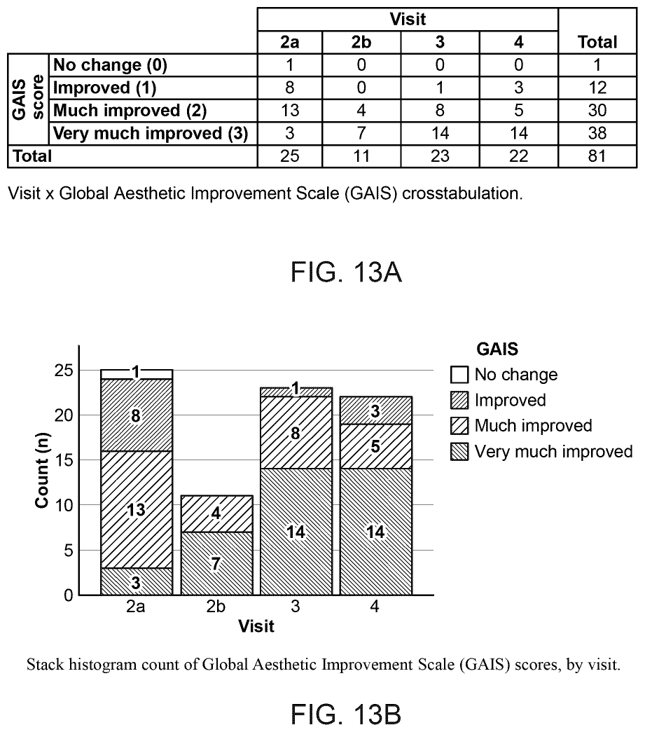

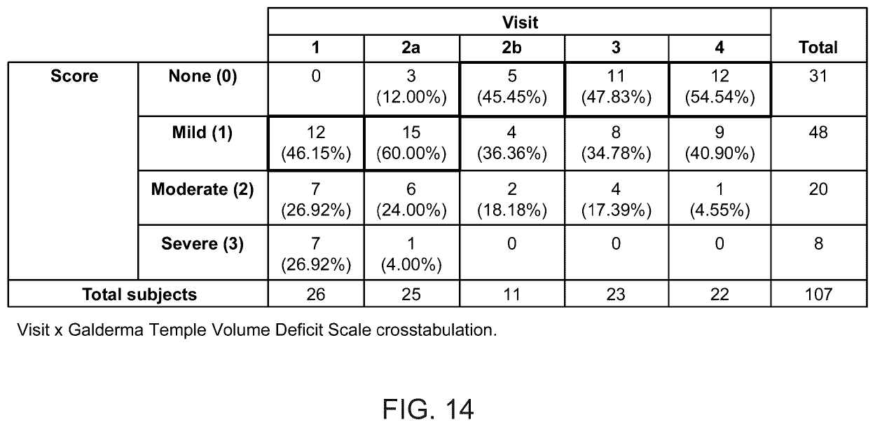

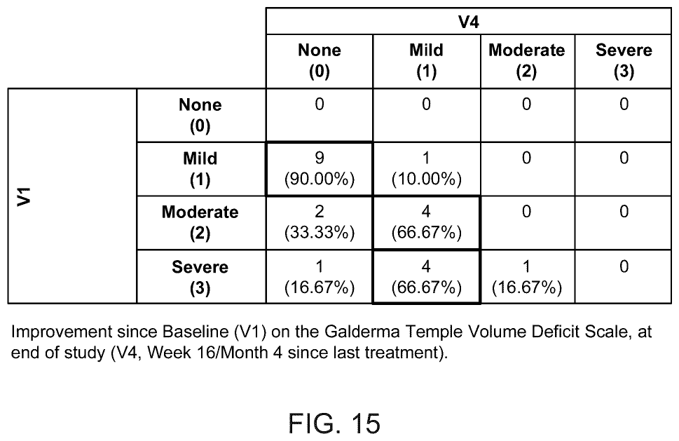

FIG. 13A is a tabular representation of GA IS for the experiment shown in FIG. 9 and FIG. 13B is a graphical representation of the GAIS. In particular, the Galderma Temple Volume Deficit Scale was assessed by a blinded evaluator. A stacked histogram is presented in FIG. 13B, displaying the count of GAIS scores per visit (FIG. 13A). Following correction, all subjects (100%) displayed improvement in their global aesthetic appearance (although it should be noted that where percentages do not sum to precisely 100%, this is due to rounding of decimal points for FIG. 13A, FIG. 14 and FIG. 15). At V4, improvement was maintained by all subjects.

FIG. 14 is a tabular representation of volume deficit scale for the experiment shown in FIG. 9, where the Galderma Temple Volume Deficit Scale was assessed by a blinded evaluator. FIG. 15 is a tabular representation of improvement since baseline for the experiment shown in FIG. 9. In FIG. 15, only data for 22 out of 26 subjects who completed the study is shown. For both such figures, Chi square tests revealed a significant effect of visit on the distribution of GTVS scores (p=<0.001, X2 (12)=37.827). Compared to V1, there were no cases of a worsening GTVS score reported throughout the study. At V4, the majority of subjects (14/22; 63.64%) displayed a 1-grade improvement, 6/22 (27.27%) displayed a 2-grade improvement, 1/22 (4.55%) displayed a three-grade improvement, and a single subject's scores remained unchanged from V1 (mild severity). Ninety percent (90%) of subjects who presented with mild temporal hollows at V1 had no temporal hollowing at V4. Subjects who presented with moderate temporal hollows at V1 had mild (66.67%) or no temporal hollowing (33.33%) at V4. Subjects who presented with severe temporal hollows at V1 had no (16.67%), mild (66.67%), or moderate (16.67%) temporal hollowing at V4, respectively. All subjects enrolled with moderate or severe temporal hollows displayed improvement at V4 (FIGS. 14 and 15).

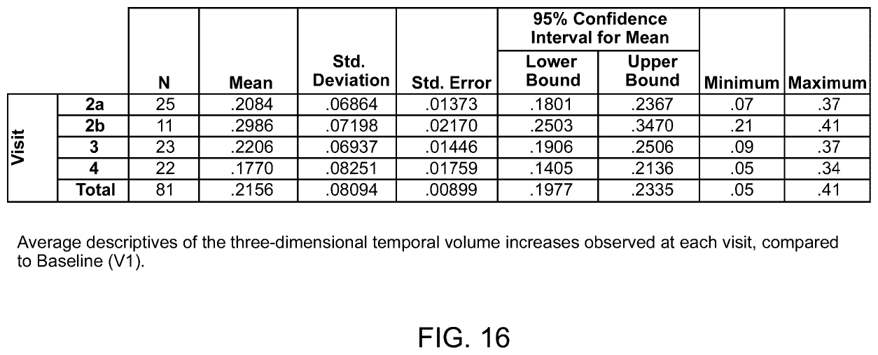

FIG. 16 is a tabular representation of average descriptors of the three-dimensional temporal volume increases observed at each visit, compared to baseline for the experiment shown in FIG. 9. The mean volume increases observed at visits 2a, 3 and 4 were not meaningfully different, and visit 2b was not compared as only a subset of subjects was observed at this point. Multiple comparisons (A NOVA) did not reveal any statistically significant difference between the values of volume change on the right versus left sides (F (3, 77)=0.950, p=0.421), when stratified by visit number. Therefore, data for each unilateral sides was combined and the average volume change for the subject, per visit, was used for the analysis. After desired correction, the maximum average increase in volume was observed at V3 (0.2206 cc, SD: 0.06937), with results maintained at V4 (0.1770 cc, SD: 0.08251; FIGS. 10 and 11). Multiple comparisons (A NOVA) did not reveal any significant decrease in volume between V3 and V4 (p=>0.05; FIG. 16).

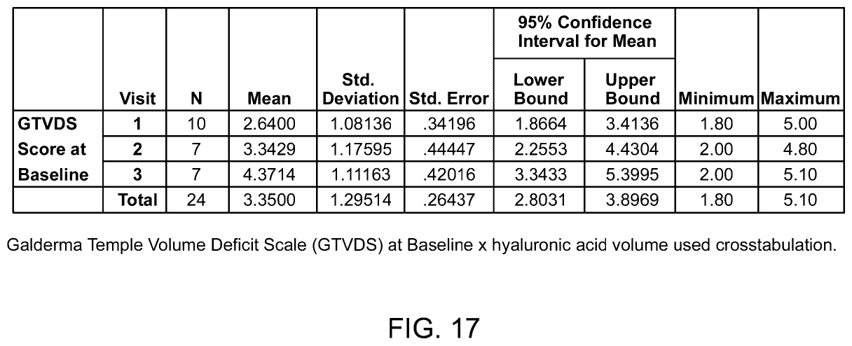

FIG. 17 is a tabular representation of Temple Volume Deficit Scale (TVDS) at Baseline x hyaluronic acid volume used cross-tabulation for the experiment shown in FIG. 9. ANOVA revealed a significant association between Baseline GTVDS scores and the amount of HA filler required to achieve desired correction, with greater temporal hollow severities at Baseline associated with the use of greater mean volumes throughout the study duration (F (2,21)=4.941, p=0.017; FIG. 17). Although the study was not powered to determine statistical significance of the below effects, the following data trends were observed: Subjects requiring two treatments received greater mean volumes of HA filler (4.31 cc, SD: 1.02) compared to subjects treated at a single visit (2.50 cc, SD: 0.87). The mean volume equated to almost a two-syringe difference (i.e., 1.81 cc). Post-menopausal women required a greater mean total volume of HA for achieving desired correction (3.67, SD: 1.28) compared to pre-menopausal women (2.81, SD: 1.19). The mean difference equated to almost a one-syringe difference (i.e., 0.86 cc).

The example experiment demonstrated that with a proper treatment technique, the risk of vascular AEs can be significantly decreased. In the associated clinical trial, over 100 bilateral aspirations were performed in twenty-six subjects, and not a single positive aspiration was observed.

The techniques of the present disclosure may provide for more informed treatments to be carried out, that can ameliorate the likelihood of adverse events. M any of the severe AEs reported in the literature are due to accidental intravascular injection of HA into one or more blood vessels or one or more arteries laying within the superficial to mid-level layers of the temporal fossa, such as the superficial temporal artery, middle temporal artery, and the zygomatico-orbital artery. Further, other AEs may be associated with accidental injection of HA into one or more nerves. By injecting deeply, even common procedural-related AEs such as swelling can be more limited and may occur less frequently than when HA-V was used in other anatomical areas and treatments were performed by the same injector. This observation may be attributable in part to local anatomy, as deep injections performed in the temporal region may cause swelling to occur deep or even within muscle. Such injections may not present any undesired aesthetic appearance or undesirable sensation to the subject. Thus, the specific treatment technique can leverage a product side effect to clinical benefit. Hence, providing instructions to a care provider to carry out such techniques can aid in achieving the above noted benefits. A skilled care provider may already possess heuristics to guide particular treatment decisions, whereas instructions of the nature contemplated herein may be to particular benefit for more novice providers.

In an example clinical trial, HA-V was found to be a suitable HA filler for volumization of the temporal region. HA-V was evidenced to have an excellent safety profile and proven efficacy up to 16 weeks. Moreover, subject satisfaction was high, with 91.3% of subjects being satisfied with the appearance of their temporal regions following correction.

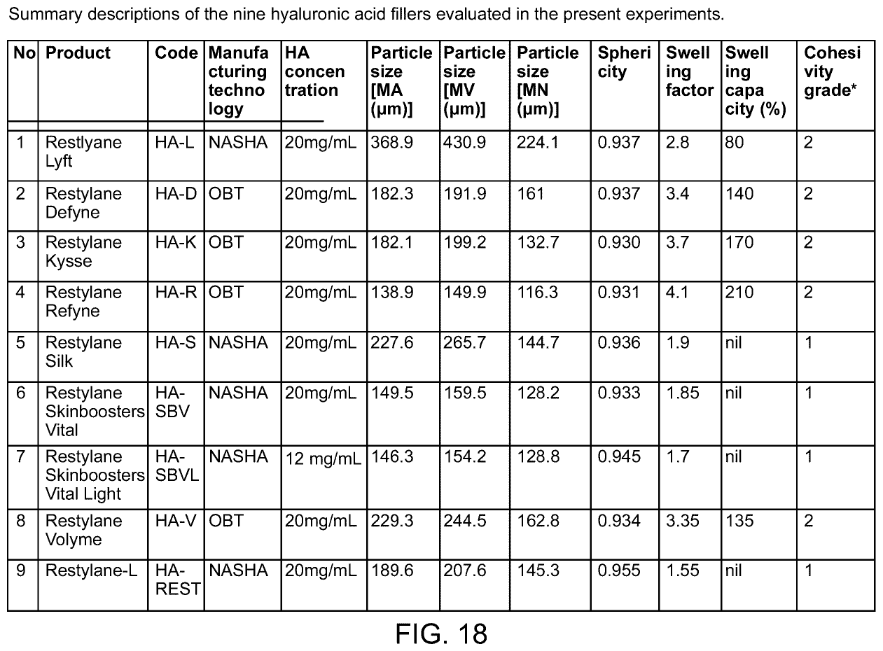

FIG. 18 is a tabular representation showing an experimental setup for an example clinical trial for in vitro hyaluronic acid. For the treatment of age-related volume loss and wrinkles, the aesthetic practitioner has a myriad of hyaluronic acid (HA) fillers to choose from. Properties that help qualify the use of certain HA fillers for specific indications have been defined and measured. Important features of HA include its hydrophilicity (ability to retain water), cohesivity (the force of attraction that holds molecules of a given substance together), and physical properties (e.g., particle size (mean and distribution), shape). In vivo, these characteristic features undergo changes following injection and are responsible for the unique behavior of the injectate (e.g., lifting versus contouring), as well as its duration of effect.

Four related experiments were performed. For each test, products from several batches were used. Experiments 1 and 2 were performed in duplicate, Experiment 3 once, and Experiment 4 three times.

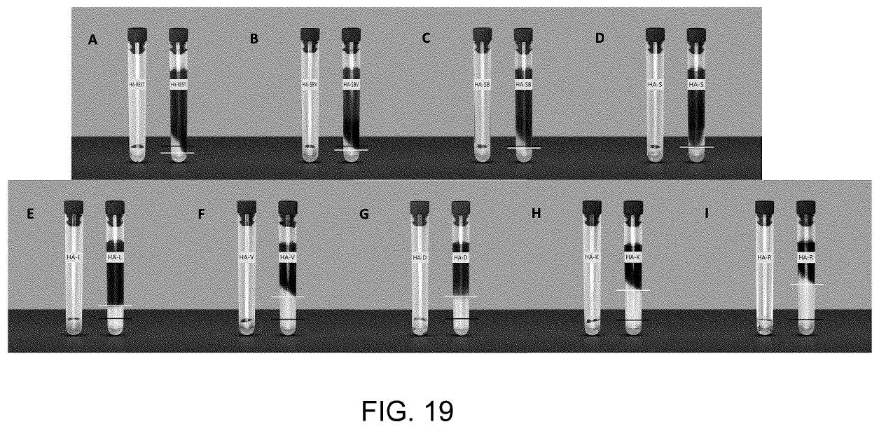

In the first experiment, nine fillers (FIG. 18) commercially available in the Canadian market were loaded from their prepackaged syringes into 10 mL test tubes, together with normal saline in a 1:7 ratio. To ensure sufficient hydration, 3.5 mL of saline was first delivered to the test tube, followed by 1 mL of HA, and topped with another 3.5 mL of saline. The low HA to saline ratio was chosen to ensure there would be enough space in the tube to visualize the interface between the saline and hydrated HA, even if large absorption rates were observed (i.e., up to 700%). The volumes used were also limited by the size of the 10 ml vials. The tubes were sealed with a screw top, inverted ten times, then left to rest at a room temperature (21.5-23.5° C.) for seven days. The room temperature was checked once daily. After seven days of incubation, the test tubes were centrifuged for five minutes at 10,000 revolutions per minute (rpm). This allowed the denser hydrated HA to pass to the bottom of the test tube, while the unabsorbed saline rose to the top. The settings were chosen based on two previous studies. The first was an experiment which allowed for approximately 35,000 rpm for ten minutes. In a later study, investigators rotated the centrifuge at 1,200 rpm for twelve minutes. Since the centrifuge rotated at the same speed as that used in the first study, the centrifuging time was reduced to five minutes. After centrifuging, 10 μm of blue dye (DipQuick counter stain #3) was pipetted onto the top surface of the sample. Its diffusion to the interface between the saline and the hydrated HA created a demarcation line. After approximately 90 minutes, two-dimensional photographs were taken to illustrate the saline-hydrated HA interface and used for analyses (FIG. 19). The swelling capabilities of each HA were quantified based on the percent increase in volume from the original 1 mL deposited into the vial, as well as the swelling factor [defined as the maximum capacity to take up additional fluid at equilibrium, determined as final ml/g and calculated by V₁/V₁, where V₁ is the initial volume of the gel at Day 1 and V₇ is the volume of the fully swollen gel at Day 7. For statistical analysis, correlations between swelling capability and other rheology properties were investigated.

A cohesivity assay was performed under standardized testing conditions, including a room temperature between 22.5-23.5° C. First, 0.1 mL of blue dye (DipQuick counter stain #3) was added as a coloring agent to 1 mL of HA gel, using a Luer Lock. After mixing the HA and dye for 1 minute, samples were loaded into an 800-ml glass beaker containing 370 ml of distilled water and a 2 cm magnetic bar stirrer, from a fixed height of 2 cm above the water surface. Each sample was loaded using the needle provided in the manufacturer's packaging. The gel and water mixture was stirred at a constant rotational frequency of 160 rpm. Standardized digital images were collected 90 seconds after complete extrusion of each sample into the beaker and commencement of magnetic stirring. The cohesivity of each specimen was assessed visually from these images, understood as the ratio of intact to dispersed gel. Two independent raters blinded to product selection graded all imagery, based on the five-point visual Gavard-Sundaram Cohesivity Scale (GSCS). The GSCS rates cohesivity from fully dispersed (1) to fully cohesive (5). A third independent and blinded rater resolved any discrepancies.

The FLOWSY NC (Microtrac MRB, Pennsylvania, USA) hybrid device was used to perform the particle size and shape analyses, using tri-laser diffraction/light scattering and image analysis measurements. For characterizing particle size, the FLOWSY NC reports three values: i) “MA” is the mean diameter of the area distribution, ii) “MV” is the mean diameter of the volume distribution, and iii) “MN” is the mean diameter of the number distribution. All values are reported (FIG. 18). For sample preparation, 0.2 mL of each filler was loaded into a 10 mL vial using the needle provided in the manufacturer's packaging. Two milliliters of distilled water were added to each vial before samples were pipetted into FLOWSYNC's water bath, prefilled with carrier fluid (200 ml distilled water). The FLOWSYNC's automated filling, de-aerating, pre-circulating, and circulating operations were used to ensure consistency and repeatability of samples. Wet operation settings included: Number of rinses: 1; Flow rate: 55%; De-aeration cycles: 3; Ultrasonic power (W): 40%; Ultrasonic time: 60 seconds. During the analysis, particles flowing in the system's stream were backlit by a high-speed strobe light and photographed by a high-resolution digital camera. The subsequent particle size and shape analyses were conducted using the system's internal hardware (Microtrac FLOWSYNC; Leeds & Northrup, St. Petersburg, FL).

In the fourth experiment, non-hydrated HA was compared to its hydrated counterpart using light microscopy. For wet sample preparation, 0.2 mL of product was loaded onto a glass slide. Samples were taken from either a new syringe (for non-hydrated HA) or the test tubes from Experiment 1 (for hydrated HA). To ensure only hydrated HA was removed from the test tubes, an 18G needle was inserted into the sample until it touched the bottom of the vial. The pre-packaged syringes that came with each product could not be used for this part of the experiment, as the suction force required for some samples was too high, likely the result of particle size expansion in the presence of saline. A new syringe and needle was used for preparing each sample, to avoid cross-contamination. A 0.2 mL drop of 0.06% blue dye (DipQuick counter stain #3, diluted with saline) was applied to the sample for 30 to 45 seconds, before being rinsed twice with 1 mL saline (total 2 mL used). A slide cover was placed over the sample and lightly pressed to spread the material, creating a thin layer suitable for microscopic evaluation. The slide was then placed under the microscope (AmScope M 170C-E 40X-1000X Dual LED Portable Compound Microscope with Camera) and the sample was examined using increasing levels of magnification. The potential correlations between swelling factor, cohesivity, and particle size were investigated.

Based on the findings of Experiment 1, the following HA fillers did not expand as they bound water: HA_{REST}, HA_{SBVL}, HA_{SBV}, HA_{S} (FIG. 19, parts A-D). Each of these fillers were manufactured using NASHA technology. The fillers that did expand (swell) as they bound water included HA_{L}, HA_{D}, HA_{V}, HA_{K}, and HA_{R} (FIG. 19, parts E-I). Except for HA_{L}, all of these fillers were manufactured using OBT technology. HA_{L} was the only NASHA product that displayed a positive swelling capacity, and of note, it is a large-particle HA. The swelling factor (ml/g) and capacity (%) of each filler is displayed in FIG. 24. In FIG. 24, the swelling factor (ml/g) and capacity (%) of each filler was calculated by V₇/V₁, where V₁ is the initial volume of gel at Day 1 and V₇ is the volume of gel at Day 7. Products with a swelling factor between 1 and 2 ml/g resulted in negligible swelling capacities (i.e., HA_{REST}, HA_{SBVL}, HA_{SBV}, HA_{S}). Swelling factors ranged from 1.55 to 4.1 ml/g and swelling capacities ranged from nil to 210%. Of all products evaluated, HA_{REST} demonstrated the least potential for swelling upon hydration with saline and HAR displayed the highest.

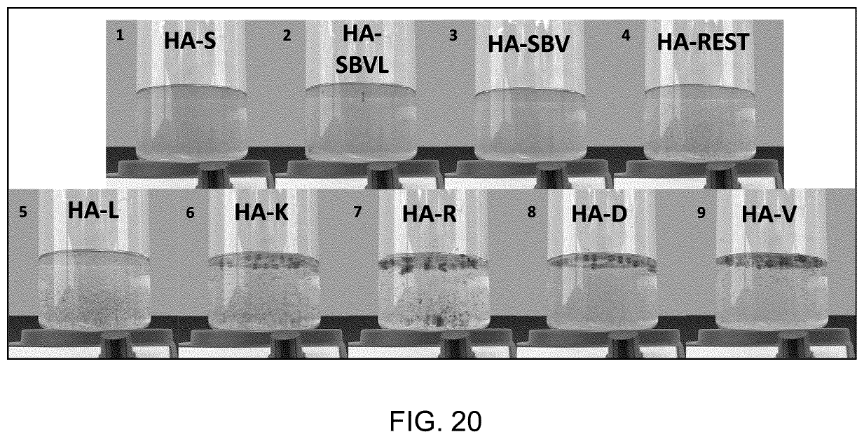

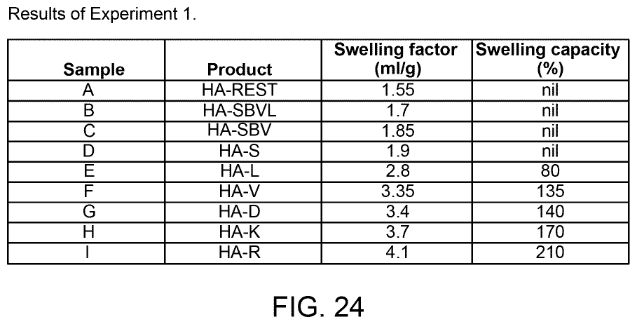

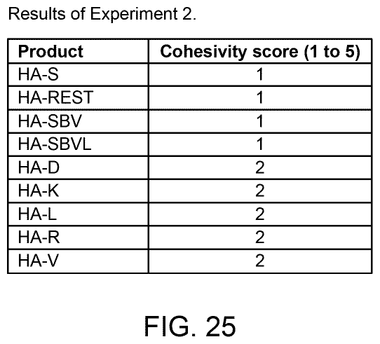

The results of Experiment 2 are displayed in FIG. 20. Based on FIG. 20, the rank order from least to most cohesive was: HA_{S}, HA_{SBVL}, HA_{SBV}, HA_{REST}, HA_{L}, HA_{K}, HA_{R}, HA_{D}, HA_{V}. Of note, HA_{SBV}, HA_{SBVL}, and HAS appeared nearly identical on the cohesivity test. Based on blinded review (FIG. 25), samples consisted of GSCS scores of 1 (fully dispersed) and 2 (mostly dispersed). In particular, FIG. 25 depicts the cohesivity of each of the nine HA products as evaluated by three blinded reviewers based on the Gayard-Sundaram Cohesivity Scale (Sundaram, 2015). None of the samples had a cohesivity score of 3 (partially dispersed-partially cohesive), 4 (mostly cohesive), or 5 (fully cohesive). It was observed that in general, NASHA products had a lower degree of cohesivity compared to products manufactured by OBT.

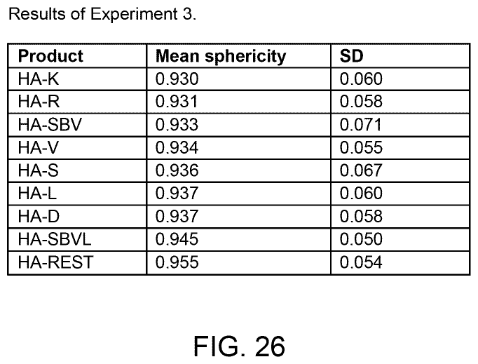

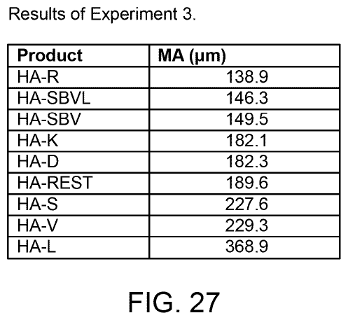

Each sample consisted of material containing particle sizes well within the FLOWSY NC's range of sensitivity (i.e., 0.01 μm to 4000 μm). Sphericity, which is a measure of how closely a particle resembles a perfect sphere (perfect sphericity=1.0) was evaluated for each sample. For all samples, the sphericity values were consistently about 1 (FIG. 26). In particular, FIG. 26 depicts the results of Experiment 3, where the mean sphericity of the particles of each sample were evaluated, with a value of 1 being a perfect sphere. FIG. 27 depicts the results of Experiment 3 with tabulation of the mean particle size (MA), where MA is the mean diameter of the area distribution.

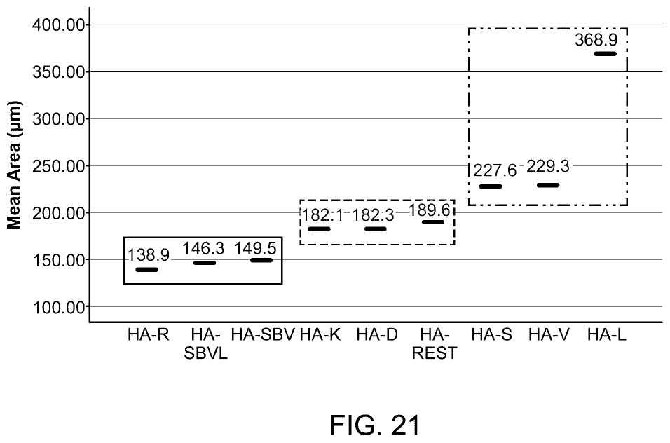

Based on the calculation of mean particle size, the mean particle size of each sample was determined to range from 138.9 to 368.9 μm (FIG. 26). From smallest to largest mean particle size, the following rank order was observed: HAR, HASBVL, HASBV, HAK, HAD, HAREST, HAS, HAV, HAL. Based on the mean particle size, three distinct subgroups were evident, including small (M: 144.9 μm; SD: 5.43), medium (M: 184.66 μm; SD: 4.27), and large-particle (275.25 μm; SD: 81.09) HA gels (FIG. 21). There was no statistically significant association between manufacturing technology and mean particle size (p>0.05), and the 95% confidence interval for both products significantly overlapped (NASHA=115.71 to 295.88; OBT=124.39 to 241.90). Based on the findings displayed in FIG. 26 and FIG. 21, the following observations were noted: HA-SBV and HA-SBVL appeared similar in size, HA-S was distinguishable from HA-SBV and HA-SBVL, HA-R (small) and HA-L (large) were on opposite ends of the particle size spectrum, HA-K, HA-D, and HA-REST were of moderate particle size. In addition to HA-L, HA-V and HA-S consisted of relatively large particles.

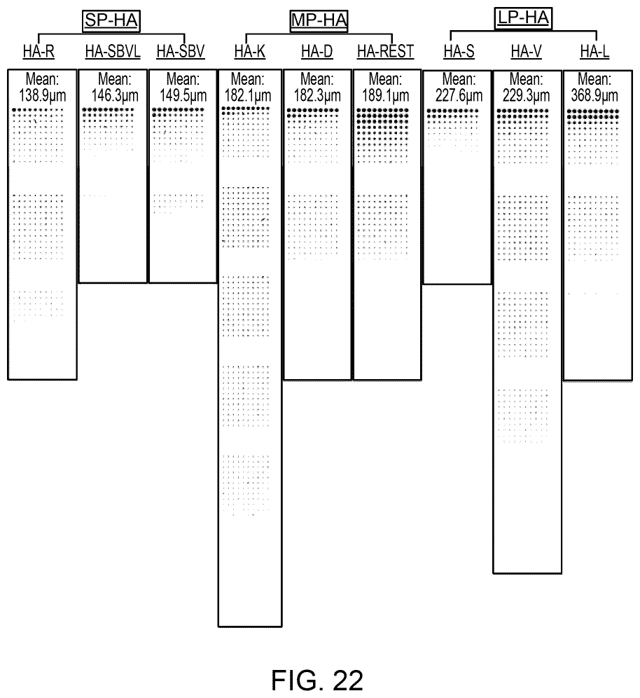

HASBVL, HASBV, HAD, HAREST, HAs, and HAL displayed a narrow range of particle sizes and HAR, HAK, and HAV displayed comparatively wider ranges. In general, products manufactured with OBT resulted in samples with a wider range of particle sizes, compared to those manufactured with NASHA. Of all samples, HAS displayed the narrowest range of particle sizes and HAK displayed the widest (FIG. 22).

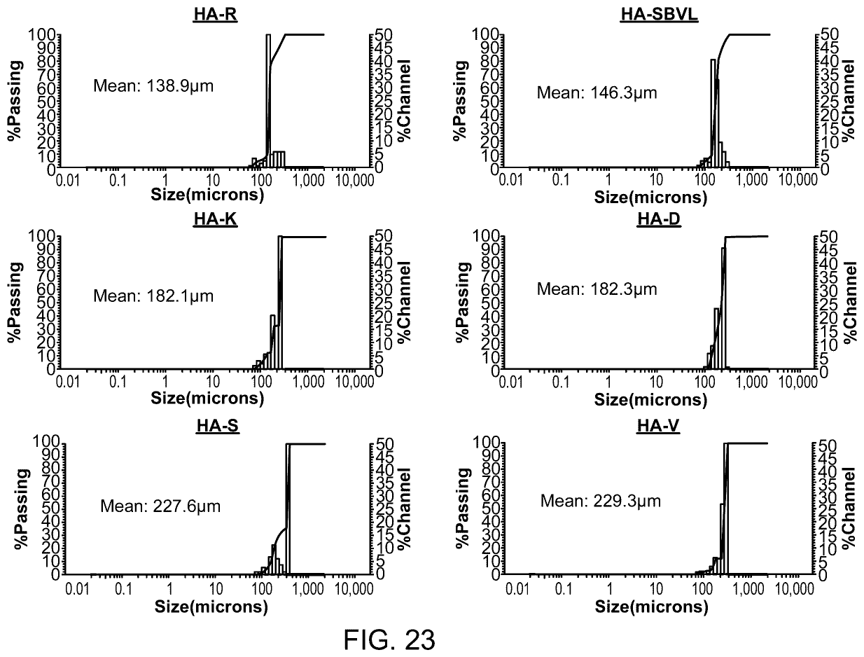

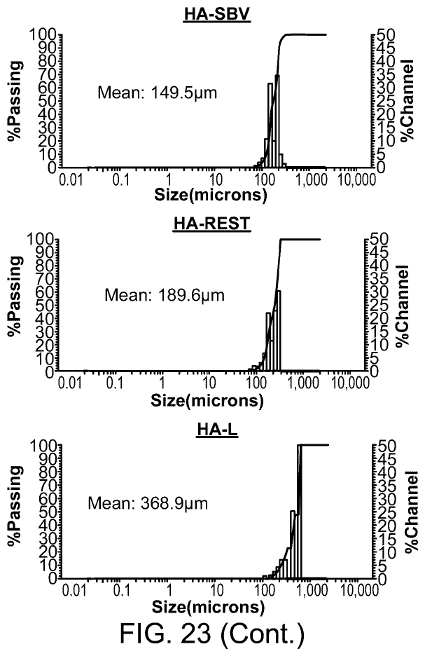

The particle size distributions are displayed in FIG. 23. A simple linear regression was calculated to predict percent (%) pass, based on product and sieve size, b (product)=0.007, b (sieve)=0.813; t (2)=2.925, p=0.004. A significant regression equation was found [F (2, 657)=640.856, p<0.001, with an R2 of 0.661, indicating that the regression model was better at predicting the % pass than the mean alone (ANOVA p<0.001). Although all samples contained variations of particle sizes, the distributions were far from normally distributed, and all samples displayed one or more clear peaks.

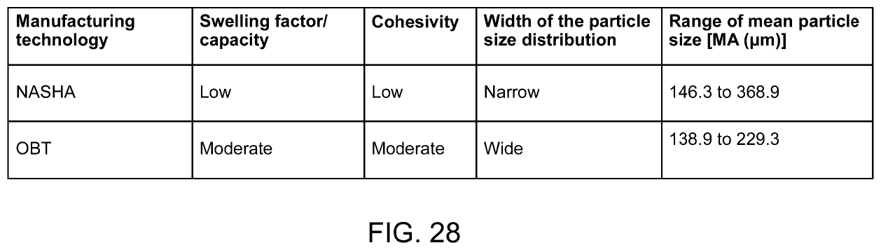

A summary of findings comparing products manufactured with NASHA versus OBT is depicted in FIG. 28. FIG. 28 shows the correlation between swelling factor and cohesivity. The swelling factor was calculated by V₇/V₁ as described above, and the cohesivity refers to the force of attraction holding the molecules of the sample together. Based on Experiments 1 through 4, it was concluded that NASHA has a low propensity for swelling, as well as a low degree of cohesivity. Conversely, OBT produces products with a higher propensity for swelling and a moderate level of cohesivity. Both methods of manufacturing resulting in HA products consisting of a range of particle sizes (i.e. 138.9 to 368.9 μm).

There was a strong positive correlation between swelling factor and degree of cohesivity (R2=0.87). This is visually evident by comparing FIGS. 18 and 19. In each Figure, the four samples presented on the top row are the same, as well as the 5 samples on the bottom row of each image. While there are a few deviations between Figures in the exact rank order of the samples, samples A to D in FIG. 19 correspond to samples 1 to 4 in FIG. 20, and samples E to I in FIG. 19 correspond to samples 5 to 9 in FIG. 20. Labels were created for each sample, based on their unique combination of swelling capacity and degree of cohesion (FIG. 29). FIG. 29 shows alphanumeric labels assigned to each sample based on the combination of swelling factor and degree of cohesion, with letters A to I representing increasing swelling factor values and numbers 1-9 representing values of increasing cohesion.

Particle size did not correlate with swelling factor or cohesivity (p>0.05). However, the width of the particle size distribution (narrow versus wide) significantly correlated with both cohesivity and swelling factor (p<0.05), with wider distributions (i.e. OBT products) associated with greater cohesivity and swelling capacity.

The findings of Experiment 1 indicate that in general, NASHA products do not have significant swelling capacities while OBT products do. This indicates that NASHA products have already reached the equilibrium of their swelling potential following the manufacturing process, and therefore have the lowest capacity to take up additional fluid. The only exception to this rule was the large-particle HA (HA_{L}), although it still had less swelling capacity than all the OBT products. The maximum swelling capacity observed in this experiment was HAR, which expanded to 210% its original volume. While this increase in volume may seem rather substantial, this represents only a moderate level of swelling relative to the swelling capabilities of fillers manufactured with different technologies.

In Experiment 2, the cohesivity of each of the nine HA fillers under evaluation was demonstrated. The results of this experiment support that the fillers ranged only slightly in degree of cohesivity, from being fully dispersed to partially dispersed (i.e., scores of 1 and 2 on the GSCS). Compared to fillers created using different manufacturing technologies (e.g., Vycross), the HA fillers evaluated did not possess a high degree of cohesion. Nonetheless, in general NASHA products had a lower degree of cohesivity compared to products manufactured by OBT.

When reviewing the results of the particle size analysis (Experiment 3), shape must be considered along with mean size and width of the distribution. Parameters related to particle morphology (e.g., sphericity) are informative as to regarding the behavior of the HA gels, (REF), and these key properties can change drastically with no significant differences reported in the results of the laser diffraction (i.e., mean size and size distribution). In addition, production conditions can fuse particles together and cause them to deviate from their desired spherical shape. Such defective particles are problematic because they have a negative effect on the flow behavior of an HA gel during injections. Therefore, the finding that all samples contained particles with a high mean sphericity value was promising.

In the literature, typically only mean particle sizes are reported and used to classify products. However, the results of a linear regression supported that particle size distributions offer better predictive value for product identification. Generally speaking, harder gels with a high G′ are more difficult to inject and require a greater extrusion force during injections. Therefore, a variety of physicochemical modifications are necessary to facilitate injection while maintaining implant persistence, such as adding un-crosslinked HA to offset injection difficulty.

The positive correlation between swelling factor and cohesion was confirmed by the present experiments. Using drop weight as a measure of cohesion, investigators found an R2 value of 0.96, which was slightly higher than the present findings of R2=0.87. However, this is likely due to the fact that their calculation of cohesivity was based on a continuous variable (i.e., weight in mg) while the present clinical trial used an ordinal scale (i.e., cohesivity grades). Interestingly, despite employing different methods to investigate product rheology, the findings are in agreement with each other: cohesive products are further away from their equilibrium of swelling and therefore, possess a greater propensity for swelling.

The findings of these four related experiments can be used by injectors as a guide to selecting the appropriate filler, based on synchrony between the products attributes (e.g., hygroscopy, particle size (mean, distribution) and shape, manufacturing technology, HA content, level of crosslinking, viscosity, cohesivity, G′ prime (or gel hardness), resistance to deformation (G*), elastic modulus (G′), viscous modulus (G″), phase angle (δ), tissue integration, lift capacity), treatment indications (including a consideration of anatomical location and depth of injection), and subject characteristics (e.g., skin thickness, skin elasticity, degree of correction required). For example, fillers shown to absorb the least amount of water could be used in areas prone to swelling, such as the periocular area and upper lip rhytids, or they could be used for more superficial injection into the dermis (e.g., HASBV, HASBVL). Moreover, as water uptake can affect an injector's ability to sculpt and integrate HA into the tissues, the findings of this study may provide additional information regarding the necessary injection target to employ.

### Exemplary Injected Compositions

In some embodiments, the compositions used for a filler described above can include hyaluronic acid. In some embodiments, the hyaluronic acid (HA) is a gel. In some embodiments, the hyaluronic acid is avian HA, bovine HA, non-animal stabilized HA, or combinations thereof. In some embodiments, the hyaluronic acid is a gel generated by a Streptococcus species of bacteria, chemically cross-linked, stabilized, and suspended in saline. Alternatively, or in combination, additional biologic, biodegradable fillers are used. Biologic, biodegradable fillers include materials derived from organism, human, and/or animal tissues and/or products.

As described herein, the HA products can include various Restylane® products. These products can include Restylane® FY NESSE, Restylane® REFY NE, Restylane® VOLYME, Restylane® KYSSE™, Restylane® DEFY NE, Restylane® LYFT™, and Restylane®.

Restylane® FY NESSE is a sterile, biodegradable, viscoelastic, non-pyrogenic, clear, colorless, and homogeneous soft hyaluronic acid gel with a moderate lifting capacity. The product has a hyaluronic acid gel concentration of 20 mg/ml and contains 3 mg/mL lidocaine hydrochloride. Restylane® FY NESSE is indicated for injection into the mid-to-deep dermis for correction of moderate to severe facial wrinkles and folds (such as nasolabial folds) in patients.

Restylane® REFY NE is a sterile, biodegradable, viscoelastic, non-pyrogenic, clear, colorless, and homogeneous soft hyaluronic acid gel with a moderate lifting capacity. The product has a sodium hyaluronate concentration of 20 mg/mL in phosphate buffered saline at pH 7 and contains 3 mg/mL lidocaine hydrochloride. Restylane® Refyne is indicated for injection into the mid-to-deep dermis for correction of moderate to severe facial wrinkles and folds (such as nasolabial folds) in patients.

Restylane® VOLY ME is a cross-linked, non-animal hyaluronic acid gel used to add volume and definition in the face in areas such as the cheekbones, cheeks, and jaw. Restylane® VOLY ME is crosslinked with BDDE (1,4-butanediol diglycidylether) to form a soft gel. Restylane® VOLY ME is used as a dermal filler and is suitable for injection into the subcutaneous layer to restore or enhance facial volume or to re-contour facial features of patients.

Restylane® KY SSE is a sterile, biodegradable, viscoelastic, non-pyrogenic, clear, colorless, flexible and homogeneous gel composed of hyaluronic acid of bacterial origin, with a moderate lifting capacity. Restylane® KY SSE is crosslinked with BDDE (1,4-butanediol diglycidylether). The product has a sodium hyaluronate concentration of 20 mg/mL in phosphate buffered saline at pH 7 and contains 3 mg/mL lidocaine hydrochloride. Restylane® KY SSE is indicated for injection into the lips for lip augmentation and the correction of upper perioral rhytids in patients.

Restylane® DEFY NE is a sterile, biodegradable, viscoelastic, non-pyrogenic, clear, colorless and homogeneous soft hyaluronic acid gel. Restylane® DEFY NE is crosslinked with BDDE (1.4-butanediol diglycidylether). The product has a sodium hyaluronate concentration of 20 mg/mL in phosphate buffered saline at pH 7 and contains 3 mg/mL lidocaine hydrochloride. Restylane® DEFY NE is indicated for injection into the mid-to-deep dermis for correction of moderate to severe, deep facial wrinkles and folds (such as nasolabial folds) in patients. Restylane® DEFY NE is indicated for injection into the mid-to deep dermis (subcutaneous and/or supraperiosteal) for augmentation of the chin region to improve the chin profile in patients with mild to moderate chin retrusion.

Restylane® LY FT is a sterile gel of hyaluronic acid generated by Streptococcus species of bacteria, chemically cross-linked with BDDE, stabilized and suspended in phosphate buffered saline at pH=7 and concentration of 20 mg/ml with 0.3% lidocaine. Restylane® Lyft with Lidocaine is indicated for implantation into the deep dermis to superficial subcutis for the correction of moderate to severe facial folds and wrinkles, such as nasolabial folds. Restylane® Lyft with Lidocaine is indicated for subcutaneous to supraperiosteal implantation for cheek augmentation and correction of age-related midface contour deficiencies in patients. Restylane® Lyft with Lidocaine is indicated for injection into the subcutaneous plane in the dorsal hand to correct volume deficit in patients.

Restylane® is a gel of hyaluronic acid generated by Streptococcus species of bacteria, chemically crosslinked with BDDE, stabilized and suspended in phosphate buffered saline at pH=7 and concentration of 20 mg/mL. Restylane® is indicated for mid-to-deep dermal implantation for the correction of moderate to severe facial wrinkles and folds, such as nasolabial folds. Restylane® is indicated for submucosal implantation for lip augmentation in patients.

### Processor Implementations

The processor 105 may include a microprocessor, an application-specific integrated circuit (A SIC), a field-programmable gate array (FPGA), etc., or combinations thereof. The memory 110 may include, but is not limited to, electronic, optical, magnetic, or any other storage or transmission device capable of providing the processor 105 with program or machine instructions. The memory 110 may include a floppy disk, compact disc read-only memory (CD-ROM), digital versatile disc (DVD), magnetic disk, memory chip, read-only memory (ROM), random-access memory (RAM), Electrically Erasable Programmable Read-Only Memory (EEPROM), erasable programmable read only memory (EPROM), flash memory, optical media, or any other suitable memory from which the processor 105 can read instructions. The instructions may include code from any suitable computer programming language such as, but not limited to, ActionScript®, C, C++, C #, HTML, Java®, JavaScript®, Perl®, Python®, Visual Basic®, and XML.

While this specification contains various implementation details, these should not be construed as limitations on the scope of what may be claimed, but rather as descriptions of features specific to particular implementations. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated in a single software product or packaged into multiple software products embodied on tangible media.

References to “or” may be construed as inclusive so that any terms described using “or” may indicate any of a single, more than one, and all of the described terms.

Various numerical values herein are provided for reference purposes only. Unless otherwise indicated, all numbers expressing quantities of properties, parameters, conditions, and so forth, used in the specification and claims are to be understood as being modified in all instances by the term “approximately.” Accordingly, unless indicated to the contrary, the numerical parameters set forth in the following specification and attached claims are approximations. Any numerical parameter should at least be construed in light of the number reported significant digits and by applying ordinary rounding techniques. The term “approximately” when used before a numerical designation, e.g., a quantity and/or an amount including range, indicates approximations which may vary by (+) or (−) 10%, 5%, or 1%.

As will be understood by one of skill in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

The claims should not be read as limited to the described order or elements unless stated to that effect. It should be understood that various changes in form and detail may be made by one of ordinary skill in the art without departing from the spirit and scope of the appended claims. All implementations that come within the spirit and scope of the following claims and equivalents thereto are claimed.

What is claimed is:

1. A method for administering an injectable filler to correct an aesthetic deficit in the temporal region of a face, the method comprising:
determining the aesthetic deficit in the temporal region of the face, wherein the aesthetic deficit is one of a deficit in lift or a deficit in volume;
selecting an injectable filler, wherein the injectable filler is a hyaluronic acid gel filler selected from one of a group I filler or a group II filler, wherein
(i) the group I filler comprises hyaluronic acid gel characterized by an X strain of about 500% to about 2500% and a G′ from about 0 Pa to about 300 Pa; and
(ii) the group II filler comprises hyaluronic acid gel having an X strain of about 0% to about 500% and a G′ from about 500 Pa to about 800 Pa; and
administering the determined group I filler as an injection at depth to correct the determined aesthetic deficit of a deficit in lift, or administering the determined group II filler as an injection at depth or as a superficial injection to correct the determined aesthetic deficit in volume.

2. The method of claim 1, wherein the group II filler comprises hyaluronic acid gel characterized by an X strain of about 750% to about 1600% and G′ of about 10 Pa to about 200 Pa.

3. The method of claim 2, wherein the determined group II filler comprises hyaluronic acid gel (i) characterized by an X strain of about 800% to about 1000% and G′ from about 100 Pa to about 200 Pa to correct the determined aesthetic deficit in volume, and wherein the injection is a superficial injection, or (ii) characterized by an X strain of about 1400% to about 1600% and G′ from about 10 Pa to about 100 Pa to correct the determined aesthetic deficit in volume, and wherein the injection is an injection at depth.

4. The method of claim 1, wherein the group I fillers comprise hyaluronic acid gel characterized by (i) an X strain of about 0% to about 100% and G′ from about 700 Pa to about 800 Pa, or (ii) an X strain of about 0% to about 100% and G′ from about 500 Pa to about 600 Pa.

5. The method of claim 1, wherein the injection at depth is proximate to the periosteum and the superficial injection is between skin (layer 1) and subcutaneous fat (layer 2).

6. A method for mitigating adverse effects of an aesthetic injection, the method comprising:
receiving by a user input to a user interface, information of a subject;
identifying one or more injection factors for injection, wherein the one or more injection factors comprise:
(i) a facial region of the subject to be modified;
(ii) an aesthetic deficit in the facial region to be modified, wherein the aesthetic deficit is selected from one of lift and volume; and
(iii) severity of the aesthetic deficit, wherein the severity is selected from one of mild, moderate, and severe;
identifying, based on the identified aesthetic deficit and the identified severity of the aesthetic deficit, a hyaluronic gel filler to be injected;
determining, based on the at least one of the identified injection factors, a landmark of the facial region for injection, wherein the landmark of the facial region for injection comprises a reduced proximity to a blood vessel or a nerve; and
providing an indication of the landmark for injection.

7. The method of claim 6, wherein the hyaluronic acid gel filler is one of a group I filler comprising hyaluronic acid gel characterized by an X strain of about 500% to about 2500% and a G′ from about 0 Pa to about 300 Pa; and a group II filler comprising hyaluronic acid gel having an X strain of about 0% to about 500% and a G′ from about 500 Pa to about 800 Pa.

8. The method of claim 7, wherein (i) the group II filler comprises hyaluronic acid gel characterized by an X strain of about 750% to about 1600% and G′ of about 10 Pa to about 200 Pa, (ii) the group II filler comprises hyaluronic acid gel characterized by an X strain of about 800% to about 1000% and G′ from about 100 Pa to about 200 Pa to correct the determined aesthetic deficit in volume and wherein the injection is a superficial injection, or (iii) the group II filler comprises hyaluronic acid gel characterized by an X strain of about 1400% to about 1600% and G′ from about 10 Pa to about 100 Pa to correct the determined aesthetic deficit in volume and wherein the injection is a deep injection.

9. The method of claim 7, wherein (i) the group I fillers comprise hyaluronic acid gel characterized by an X strain of about 0% to about 100% and G′ from about 700 Pa to about 800 Pa or (ii) the group I fillers comprise hyaluronic acid gel characterized by an X strain of about 0% to about 100% and G′ from about 500 Pa to about 600 Pa.

10. The method of claim 6, further comprising providing an indication to inject the hyaluronic acid gel filler proximate to the periosteum or between skin and subcutaneous fat.

11. The method of claim 10, wherein the indication to inject the hyaluronic acid gel filler proximate to the periosteum is associated with a deficit in volume or a deficit in lift.

12. The method of claim 10, wherein the indication to inject the hyaluronic acid gel filler between skin and subcutaneous fat is associated with a deficit in volume.

13. The method of claim 6, wherein the facial region of the subject to be modified comprises at least one of temporal hollowing, skin lines, scarring of the skin, or skin folds.

14. A non-transitory computer readable medium storing instructions that, when executed by one or more processors, cause the one or more processors to perform a method, the method comprising:
receiving, by a user input to a user interface, information of a subject;
identifying injection factors for injection, wherein the injection factors comprise:
(i) a facial region of the subject to be modified;
(ii) an aesthetic deficit in the facial region to be modified, wherein the aesthetic deficit is selected from one of lift and volume; and
(iii) severity of the aesthetic deficit, wherein the severity is selected from one of mild, moderate, and severe;
identifying, based on the identified aesthetic deficit and the identified severity of the aesthetic deficit, a hyaluronic gel filler;
determining, based on the at least one of the identified injection factors, a landmark of the facial region for injection, wherein the landmark of the facial region for injection comprises a reduced proximity to a blood vessel or a nerve; and
displaying an indication of the landmark for injection.

15. The non-transitory computer readable medium of claim 14, wherein the hyaluronic acid gel filler is one of a group I filler comprising hyaluronic acid gel characterized by an X strain of about 500% to about 2500% and a G′ from about 0 Pa to about 300 Pa; and a group II filler comprising hyaluronic acid gel having an X strain of about 0% to about 500% and a G′ from about 500 Pa to about 800 Pa.

16. The non-transitory computer readable medium of claim 15, wherein (i) the group II filler comprises hyaluronic acid gel characterized by an X strain of about 750% to about 1600% and G′ of about 10 Pa to about 200 Pa, or (ii) the group I fillers comprise hyaluronic acid gel characterized by an X strain of about 0% to about 100% and G′ from about 700 Pa to about 800 Pa.

17. The non-transitory computer readable medium of claim 15, wherein the group II filler comprises hyaluronic acid gel characterized by an X strain of about 800% to about 1000% and G′ from about 100 Pa to about 200 Pa to correct the determined aesthetic deficit in volume and wherein the injection is a superficial injection.

18. The non-transitory computer readable medium of claim 15, wherein the group II filler comprises hyaluronic acid gel characterized by an X strain of about 1400% to about 1600% and G′ from about 10 Pa to about 100 Pa to correct the determined aesthetic deficit in volume and wherein the injection is an injection at depth.

19. The non-transitory computer readable medium of claim 14, wherein the method performed by the one or more processors further comprises displaying an indication to inject the hyaluronic acid gel filler proximate to the periosteum or between skin and subcutaneous fat.

20. The non-transitory computer readable medium of claim 14, wherein the method performed by the one or more processors further comprises displaying an indication of the identified hyaluronic acid gel filler.
